(19) United States
(12) Patent Application Publication
(10) Pub. No.: US 2010/0047355 A1
(43) Pub. Date: Feb. 25, 2010

(54) MAGNETIC RESONANCE-DETECTABLE, ULTRASOUND-DETECTABLE AND/OR RADIOPAQUE MICROCAPSULES AND USES THEREOF

(75) Inventors: Jeff Bulte, Fulton, MD (US); Bradley Powers, Baltimore, MD (US); Aravind Arepally, Baltimore, MD (US); Dara Lee Kraitchman, Oxford, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/226,648

(22) PCT Filed: Apr. 24, 2007
(86) PCT No.: PCT/US2007/009992

§ 371 Date: Jul. 21, 2009

Related U.S. Application Data
(60) Provisional application No. 60/794,418, filed on Apr. 24, 2006.

(51) Int. Cl. A61K 9/50; A61K 35/12; G01R 33/48

(52) U.S. Cl. 424/490; 424/93.1; 424/93.7; 324/309

(57) ABSTRACT

The present invention provides a microcapsule for implantation into a mammalian body, comprising: a) at least one cell and/or bioactive agent; and b) a biocompatible semi-permeable membrane encapsulating the at least one cell, wherein the biocompatible semi-permeable membrane comprises: at least one polycationic polymer region, at least one alginate polymer region, and a paramagnetic or superparamagnetic metal that does not participate in the crosslinking of the alginate polymer. The present invention further provides methods of making the microcapsules of this invention and use of the microcapsules of this invention in methods of delivering cells and/or therapeutic agents to a subject and in methods of embolization.

This application claims the benefit of U.S. Provisional Patent Application Ser. No. 60/794,418, filed Apr. 24, 2006, the content of which is incorporated herein by reference in its entirety.

### STATEMENT OF GOVERNMENT SUPPORT

The present invention was funded in part by government support under grant numbers EB004348, DK077537, HL073223 and NS045062 from the National Institutes of Health. The United States Government has certain rights in this invention.

### FIELD OF THE INVENTION

The present invention relates to microcapsules for the immunoisolation of cellular therapeutics and/or for use as embolic agents. The present invention also relates to methods of forming the microcapsules, compositions including the microcapsules, methods of delivering the microcapsules into mammals and apparatuses for the detection of the microcapsules.

### BACKGROUND OF THE INVENTION

Recent advances in islet cell transplantation for type I diabetes mellitus (TIDM) have provided insulin-independence in patients through successful engraftment. See, e.g., Ryan et al., Diabetes Obes Metab, 2006, 8 (1) 1-7. Islet transplantation provides a moment-to-moment fine regulation of insulin not seen with exogenous insulin injection. A recent multi-institutional trial confirmed that the so-called “Edmonton protocol” could be reproduced elsewhere with an impressive insulin-independent rate of 90%. However, success rates were found to vary, with some rates as low as 23%. See Shapiro et al., N Engl J Med, 2000, 343, 230-2381. The restricted availability of cadaveric human donor pancreata, in conjunction with potential risks associated with immunosuppression attenuate the impact of islet cell transplantation on clinical therapy of TIDM. Zwillich, Science, 2000, 289 (5479) 531-3. In addition to the health complications associated with chronic immunosuppressive therapy, many immunosuppressive regimens have been found to be selectively toxic to isolated islet cells. Drachenberg et al., Transplantation, 1999, 68 (3) 396-402. For this reason, a method of transplanting islets free of an immunosuppressive regimen would be ideal.

Microencapsulation of therapeutic cells has provided a range of promising treatments for a number of diseases including type I diabetes, hemophilia, cancer, Parkinson's disease, and fulminant liver failure. See, e.g., Ryan et al., Diabetes, 2005, 54 (7) 2060-9; Wen et al., J Gene Med, 2006, 8 (3) 362-9; Joki et al., Nat Biotechnol, 2001, 19 (1) 35-9; Chang, Panminerva Med, 2005, 47 (1) 1-9; Sajadi et al., Neurobiol Dis, 2006, 22 (1) 119-29; Mai et al., Transplant Proc, 2005, 37 (1) 527-9. Microencapsulation may create a semipermeable membrane that may prevent the passage of antibodies and complement thereby reducing or preventing graft rejection. See, e.g., Orive et al., Biomaterials, 2006, 20, 3691-700. While antibodies may be blocked, the selective permeability of the capsule may allow for passage of therapeutic factors produced by encapsulated cells. Some of the most convincing arguments for microencapsulation include the possibility of eliminating immunomodulatory protocols or immunosuppressive drugs while allowing for the long-term de novo delivery of therapeutic factors (drugs or cells) in either a local or systemic manner.

By enabling the immunoisolation of xenogenic grafts, microencapsulation could provide a means of transplanting a relatively inexhaustible source of islets, such as porcine islets, free of immunosuppresion. See Elliot et al., Transplant Proc, 2005, 37 (1) 466-9. However, the outcome of multi-institutional trials has shown that insulin-independence success rates vary widely. As the underlying differences that cause these significant variations are poorly understood, there is an urgent need for non-invasive monitoring of the fate of (encapsulated) islets following transplantation. In particular, a more sensitive means of correlating the long-term function of individual islets with the anatomical location and route of transplantation is necessary, as well as a method to assess successful engraftment and the persistence of capsule integrity when immunoprotection via encapsulation is utilized.

One such method for tracking the fate of islet cells and/or the microcapsules is via magnetic resonance imaging (MRI). Many MRI cell tracking approaches have involved labeling of cells directly, for example with superparamagnetic iron oxides (SPIOs). However, a potential concern with SPIO labeling of cells is the induction of iron overload and oxidative damage (Fenton-type reactions) by free radicals. In addition, a few reports have indicated undesirable side effects from SPIO-labeling. For instance, it was shown that Feridex®-labeled mesenchymal stem cells (MSCs) were unaltered in their viability and cell proliferation, and differentiated normally into adipocytes and osteocytes, which is their normal downstream differentiation pathway. However, at the same time, there was a dose-dependent, marked inhibition of chondrogenic differentiation. As for direct labeling of islets, it is not entirely clear whether SPIO-labeling can lead to changes in gene expression or inhibition of insulin secretion. Another limitation of long-term cellular imaging of ferumoxide-labeled cells is the resulting dilution of MR contrast when cells divide, although this may play a limited role in detection of labeled islets due to the limited amount of cell replication. Loss of islet detectability, however, may occur when labeled cells dislodge from transplanted islets and escape into the circulation or the surrounding (liver) tissue.

There have been a few examples of SPIO-containing microcapsules that are trackable by MR imaging reported in the literature. For example, a method explored by Shen et al. for incorporating a contrast agent into microcapsules, involves the use of a magnetized alginate. Shen et al., Human Gene Therapy, 2005, 16, 971-984. In the analysis of microcapsule properties that incorporate magnetized alginate, it was found that magnetic capsules had a decrease in mechanical stability as compared to non-contrast containing capsules. This was hypothesized to be due to the presence of iron aggregates in the magnetized alginate. Further, slow release of iron from the capsules was demonstrated after a period of eight months.

Thus, there remains a need in the art for compositions and methods useful in the imaging of microencapsulated transplanted cells, and in particular islet cells, either via MRI or other imaging techniques, as well as a need for embolization methods employing microcapsules.

### SUMMARY OF THE INVENTION

According to some embodiments of the invention, provided is a microcapsule for implantation into a mammalian body comprising at least one cell and/or biological or bioactive agent, e.g., a drug, chemical reagent, protein, peptide, nucleic acid, vector (viral vector), enzyme, regenerative agent (e.g., growth factor, growth modulating factor, etc.), antibody, toxin (e.g., volkesin, ricin, morrhuate, botulinum toxin, diphtheria toxin, etc.) a chemotherapeutic drug to treat a tumor or malignant cell, an immunosuppressant, a thrombolytic drug (e.g., tissue plasminogen activator (t-PA), reteplase, tenecteplase, alteplase, lanoteplase, urokinase, streptokinase, staphylokinase, etc.), a nucleic acid encoding a therapeutic protein or bioactive RNA, a vector (e.g., a viral vector), and any combination thereof, and a biocompatible semi-permeable membrane encapsulating the at least one cell and/or bioactive agent. Any suitable cell and/or bioactive agent may be encapsulated in the microcapsule of this invention. In some embodiments, the at least one cell can be, for example, an islet cell.

In some embodiments of the invention, the biocompatible semi-permeable membrane comprises at least one polycationic polymer region; at least one alginate polymer region; and a paramagnetic or superparamagnetic metal that does not participate in the crosslinking of the alginate polymer. In some embodiments, the paramagnetic or superparamagnetic metal can be iron, gadolinium, manganese, dysprosium and any combination thereof. For example, a superparamagnetic iron compound, ferum-oxide, may be used. In some embodiments, the iron compound is provided to the microcapsule via a clinical grade ferum-oxide composition, such as via a Feridex® or Resovist® colloidal solution.

In some embodiments of the invention, the biocompatible semi-permeable membrane comprises at least one polycationic polymer region; at least one alginate region; and a radiopaque contrast agent. In some embodiments, the radiopaque contrast agent includes bismuth, and in some embodiments, the radiopaque contrast agent includes barium. In other embodiments, the radiopaque contrast agent can include iodinated compounds and/or tantalum.

In some embodiments of the invention, the biocompatible semi-permeable cell membrane comprises at least one polycationic polymer region; at least one alginate region, and a fluorocarbon (or perfluorcarbon). In some embodiments, the fluorocarbon is detectable by MRI and ultrasonography, and in some embodiments, the fluorocarbon is also radiopaque. Exemplary fluorocarbons include perfluorobromides and perfluoro-crown ethers.

The present invention further provides microcapsule for implantation into a mammalian body, comprising: a) at least one cell and/or biological agent; and b) a biocompatible semi-permeable alginate layer encapsulating the at least one cell and/or biological agent, wherein the biocompatible semi-permeable alginate layer comprises a paramagnetic or superparamagnetic metal that does not participate in the crosslinking of the alginate layer. In some embodiments, the paramagnetic or superparamagnetic metal can be iron, gadolinium, manganese, dysprosium and any combination thereof. In particular embodiments, the paramagnetic or superparamagnetic metal is iron, which is present in the microcapsule as a ferum-oxide. The ferum-oxide can be derived from a Feridex® or Resovist® aqueous colloidal solution.

Also provided are compositions comprising any of the microcapsules of this invention, in a pharmaceutically acceptable carrier.

Furthermore, methods of delivering a cell and/or biological agent to a mammal (e.g., a human) comprising introducing the microcapsule according to any embodiment of the invention into the mammal, are provided herein. In some embodiments, the microcapsule is introduced by injecting the microcapsule into the mammal via a magnetic resonance-detectable needle. In addition, in some embodiments, the microcapsule is injected into the mammal, e.g., into the portal vein, the heart, the muscle, the brain, the arterial supply, etc., of the mammal, in a pharmaceutically acceptable carrier.

In addition, provided herein are methods of synthesizing microcapsules. In some embodiments, a method of synthesizing an MRI-detectable microcapsule comprises forming a droplet comprising a cell and/or biological agent, an alginate polymer that is not crosslinked with a paramagnetic or superparamagnetic metal, and at least one of a paramagnetic or a superparamagnetic metal; adding a crosslinking agent to crosslink the alginate polymer; introducing the crosslinked droplet to a polycationic polymer solution; and introducing the polycationic polymer-treated crosslinked droplet to an alginate polymer solution. Also provided is a method of synthesizing an MRI-detectable microcapsule, comprising forming a droplet comprising a cell and/or biological agent, and an alginate polymer that is not crosslinked with a paramagnetic or superparamagnetic metal; adding a crosslinking agent to crosslink the alginate polymer; introducing the crosslinked droplet to a polycationic polymer solution; and introducing the polycationic polymer-treated crosslinked droplet to an alginate polymer solution. In some embodiments, methods of forming a radiopaque microcapsule include forming a droplet comprising a cell and/or biological agent, an alginate polymer and a radiopaque contrast agent; adding a crosslinking agent to crosslink the alginate polymer; introducing the crosslinked droplet to a polycationic polymer solution; and introducing the cationic polymer-treated crosslinked droplet to an alginate polymer solution. In some embodiments of the methods of synthesis of this invention, the alginate is crosslinked with a divalent cation such as Ca²⁺, Ba²⁺, Mg²⁺, Fe²⁺, Mn²⁺ and any combination thereof.

In additional embodiments, the present invention provides a method of synthesizing an MRI-detectable microcapsule, comprising: a) forming a droplet comprising: a cell and/or biological agent, an alginate polymer that is not crosslinked with a paramagnetic or superparamagnetic metal, and at least one of a paramagnetic or a superparamagnetic metal; and b) adding a crosslinking agent to crosslink the alginate polymer. In some embodiments presented herein, the crosslinking agent can be a divalent metal cation, which can be, for example, Ca²⁺, Ba²⁺, Mg²⁺, Fe²⁺, Mn²⁺ and any combination thereof. Further, in particular embodiments, the alginate polymer solution of this method does not comprise a paramagnetic or superparamagnetic metal. In some embodiments, the droplet is formed using an electrostatic droplet generator.

The present invention additionally provides a method of embolizing a vascular site through physical obstruction, comprising introducing into the vascular site one or more microcapsules comprising a biocompatible semi-permeable membrane, wherein the biocompatible semi-permeable membrane comprises: at least one polycationic polymer region, at least one alginate polymer region, and a paramagnetic or superparamagnetic metal that does not participate in the crosslinking of the alginate polymer.

Also provided herein is a kit comprising any of the microcapsules as described herein, a syringe, and optionally instructions for using the syringe to inject the microcapsule into a mammal.

In addition, also provided herein are apparatuses for use with the microcapsules and microcapsule compositions described herein. In some embodiments, provided is an MRI system for MRI imaging of the microcapsules as described herein, comprising: an MRI scanner; a receiver configured to detect a change in magnetic resonance signal induced by the microcapsule; and a display in communication with the MRI scanner configured to display in vivo images of the microcapsules in target tissue. The MRI system may further comprise an MRI compatible delivery device releasably holding the microcapsules therein, the delivery device configured to cooperate with the MRI scanner to allow a clinician to deliver the microcapsules under an MRI guided interventional procedure.

Furthermore, also provided herein is an apparatus for imaging a microcapsule of the invention, comprising an X-ray source, and an X-ray detection device and output circuit that generates visual data associated with the location of the microcapsule in a position in the body.

### BRIEF DESCRIPTION OF THE DRAWINGS

FIGS. 1a-h. Macroscopic (a-c) and microscopic (d-h) appearance of magnetocapsules. (a) Unlabeled capsules (without Feridex®) feature a transparent appearance of alginate. (b) Unstained Feridex®-containing MR-caps exhibit a ferric rust-like color originating from the Feridex® iron oxide particles. (c) Prussian Blue (Fe³⁺-specific) staining of Feridex®-containing MR-caps. (d-f) A single human islet encapsulated without (d) and with (e, f) Feridex®. (d, e) Unstained samples, showing ferric rust color from iron oxides in (e) and uniform, smooth incorporation of iron particles without clustering or aggregation. (f) Staining with dextran-specific FITC-antibody demonstrates the presence of the dextran coat of Feridex® particles (green); islet is stained with DAPI for cell nuclei (blue). (g) Single MR-cap-containing encapsulated βTC-6 cells (dextran-(Feridex®) specific immunostaining-green; DAPI-blue). (h) Newport Green and Propidium Iodide staining of βTC-6 cells 48 h after magnetoencapsulation demonstrates >95% cell viability. Bars in (a-c) represent 1 mm and in (d-h) 150 μm.

FIG. 2. In vitro functionality of MR-cap islets is retained. Insulin secretion of encapsulated islets without Feridex® (solid bars) and MR-cap islets (open bars) in culture were measured (n=2) over a 15-day period. After the initial time point, the FDA-approved test for bioequivalence (TOST) showed no significant difference (NS) between the two capsule preparations except for the earliest time point.

FIG. 3. MRI appearance of MR-caps. (a, b) As MR-caps rapidly settled in solution, they were embedded in a 2% agarose phantom at a density of 50 capsules/ml gel. Individual MR-caps can be easily identified as hypointensities. (c, d) MR-caps before (c) and after (d) rupture using glass bead treatment. A significant loss of hypointensity can be seen. After rupture, the Feridex®-induced contrast reduces to a pinpoint double-dipole T2* susceptibility effect. (e) MR image of a mouse following injection of 500 capsules in the peritoneal cavity. Single capsules are easily identified (arrows). As a proof-of-principle experiment that the MR properties of MR-caps change when they disintegrate, they were mixed with 1 mm glass beads, shaken by hand, and then incubated on a rocker. Glass beads were removed and the disrupted beads were imaged side-by side with intact beads. The MR properties were found to change significantly following capsule rupture, with a 72% loss of hypointense Feridex® signal. This demonstrates that MRI is able to detect capsule disintegration.

FIG. 4. An exemplary laboratory set-up for the production of microcapsules according to some embodiments of the invention. An ignition wire (FIG. 4a) is connected to the van de Graaff dome (FIG. 4b). The other end is connected to a 20 g 1½″ blunt needle. The needle is fitted on a 1 cc tuberculin syringe (FIG. 4c). A Petri dish (FIG. 4d), containing isotonic (1.70%) calcium chloride dihydrate, buffered with 10 mM HEPES, is placed under the needle. A stainless wire is immersed in the calcium solution and connected to a ground. The current is adjusted by changing the van de Graaff belt speed (FIG. 4e). The islets/alginate solution is passed through the needle with a flow rate of about 200 ul/min using a nanoinjector pump (FIG. 4f).

FIG. 5. Viability of human islets encapsulated in barium x-caps (black bar), bismuth x-caps (grey bar) and alginate/poly-L-lysine (PLL)/alginate (APA) controls (white bar) after 1, 7 and 14 days in culture. *=statistically significant difference of viability as compared to control.

FIG. 6. Image of X-caps in culture and under fluoroscopy. A) Macroscopic image of bismuth x-caps. B) Fluoroscopic image of bismuth x-caps. C) Macroscopic image of barium x-caps. D) Fluoroscopic image of barium x-caps.

### DETAILED DESCRIPTION OF EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art. In the drawings, the thickness of layers and regions are exaggerated for clarity. Like numbers refer to like elements throughout. As used herein the term “and/or” includes any and all combinations of one or more of the associated listed items and may be abbreviated as “/.”

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, regions, steps, operations, elements, and/or components, but do not preclude the presence or addition of one or more other features, regions, steps, operations, elements, components, and/or groups thereof.

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

According to some embodiments of the invention, provided are microcapsules for implantation into a mammalian body that comprise at least one cell and/or bioactive agent, and a biocompatible semi-permeable membrane encapsulating the at least one cell and/or bioactive agent. There is no limitation on the type of cells that may be encapsulated, but in some embodiments of the invention, the cells are mammalian, and in other embodiments, the cells are porcine. Furthermore, in some embodiments, the cells are islet cells. Specific examples of cells include, but are not limited to islet cells, hepatocytes, embryonic stem cells, neural stem cells, neurons, glial cells and precursors, mesenchymal stem cells, fibroblasts, osteoblasts, osteoclasts, chondrocytes, immune cells (e.g., lymphocytes, monocytes, macrophages) bone marrow-derived stem cells, adipose-derived stem cells, immortalized cell lines, engineered cell lines (e.g., to produce angiostatins for tumor therapy or cytosine deaminase for chemotherapy and/or to provide prodrugs, proproteins, etc., which are not active in the microcapsule but that are activated or capable of being activated upon exposure to or entry into the extracapsular environment), epidermal stem cells, smooth muscle cells, cardiac stem cells and cardiomyocytes.

In some embodiments of the invention, the biocompatible semi-permeable membrane comprises at least one polycationic polymer region, at least one alginate polymer region and a paramagnetic or superparamagnetic metal that does not participate in the crosslinking of the alginate polymer. These embodiments may be referred to as magnetocapsules or “MR-caps.” In some embodiments of the present invention, the biocompatible semi-permeable cell membrane comprises at least one polycationic polymer region; at least one alginate region, and a radiopaque contrast agent. These embodiments may be referred to as “X-caps.”

Any suitable alginate polymer may be used in the biocompatible semi-permeable membrane. For example, the ratio of guluronate to mannuronate in the alginate may be in any proportion, such as 100:1, 50:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:50, 1:100. Furthermore, the alginate polymer may be present in a buffer solution, such as a HEPES buffer. In addition, in some embodiments, the alginate polymer solution may also include other additives such as glucose, amino acids, insulin, transferrin, serum, albumin, perfluorocarbons (PFCs) and any other component commonly found in tissue culture medium, in any combination. Examples of commercially available alginates that may be used include clinical, pharmaceutical grade alginate formulations, such as Protanal® and/or Keltone® alginates. Both Protanal® and Keltone® alginates are used as coating agents in oral medications and in food products for human consumption.

Furthermore, any suitable polycationic polymer may be used in the biocompatible semi-permeable membrane. In some embodiments, the polycationic polymer is polylysine, and in some embodiments, poly-L-lysine (PLL). In other embodiments, the polycationic polymer may be poly-L-ornithine, chitosan, polyethylene glycol and/or protamine sulfate. Protamine sulfate (PS) is clinically being used as a plasma agent to reverse heparin toxicity in anti-coagulation therapy (the polycationic protamine sulfate binds to the negatively charged heparin). A combination of different polycationic polymers may also be used. The polycation polymer may stabilize the microparticles.

Thus, in some embodiments of the invention, the biocompatible semi-permeable membrane is an alginate/poly-L-lysine (PLL)/alginate (APA) microcapsule, wherein the positively charged amino group of the lysine molecule may interact with the negatively charged carboxyl and hydroxyl groups of the uronic acid. Other alginate/polycation formulations may be used, as described, e.g., in U.S. Pat. Nos. 6,365,385, 5,084,350, 4,663,286, 5,762,959, 5,801,033, 5,573,934, 5,380,536, 5,227,298, 5,578,314, 5,693,514, 5,846,530, which contents are incorporated herein by reference in their entireties.

Any suitable paramagnetic or superparamagnetic metal may be used. In some embodiments, the metal is the superparamagnetic ferum-oxide. In some embodiments, the ferrum oxide is derived from an FDA-approved ferumoxide formulation, such as Feridex® colloidal solutions. Nonlimiting examples of other metals that may be used include gadolinium, manganese, ferric iron, dysprosium and combinations thereof.

Furthermore, in some embodiments, a combination of metals may be used. In some embodiments of the invention, the paramagnetic or superparamagnetic metal is present in the biocompatible semi-permeable membrane complexed to the polycationic polymer. For example, negatively charged ferumoxide is known to complex with PLL through electrostatic interactions, and thus, the PLL in APA capsules may complex Feridex®. In some embodiments, the paramagnetic or superparamagnetic metal is present in the biocompatible semi-permeable membrane throughout the inner alginate core. In this case, the paramagnetic or superparamagnetic metal may also interact to some extent with the polycationic polymer that is also present in the biocompatible semi-permeable membrane. An embodiment whereby paramagnetic or supermagnetic metal is included in both the core alginate region and complexed to the polycationic polymer region is also envisioned. In addition, various sizes and morphologies of the microcapsules may be formed, but in some embodiments, the microcapsules are spherical and can range in size from about 50 μm to about 1000 μm (e.g., about 50, 75, 100, 150, 200, 250, 300, 350, 400, 450, 500, 600, 700, 800, 900, or 1000 μm) and in some embodiments, can have a size of about 350 μm. In some embodiments, a reproducible synthesis may result in stable (i.e., that do not rupture) MR-caps in physiological-grade solution for at least several months (e.g., up to 12-18 months) after synthesis.

According to some embodiments of the invention, methods of synthesizing a MRI-detectable microcapsule are provided, comprising forming a droplet comprising a cell, an alginate polymer that is not crosslinked with a paramagnetic or superparamagnetic metal, and at least one of a paramagnetic or a superparamagnetic metal; adding a crosslinking agent to crosslink the alginate polymer; introducing the droplet to a polycationic polymer solution; and introducing the droplet to an alginate polymer solution. The crosslinking agent may be any agent known in the art for crosslinking alginates, such as Ca²⁺, Ba²⁺, Fe²⁺, Mg²⁺, Mn²⁺ and the like.

In this method, the paramagnetic or superparamagnetic metal is not crosslinked with the alginate, but is instead impregnated within the microcapsule and/or complexed to the polycationic polymer. Impregnating microcapsules with a stable, dextran-coated superparamagnetic metal such as Feridex® instead of de novo synthesis of uncoated superparamagnetic iron oxides such as magnetized alginate, followed by metal crosslinking of the alginate, can have a number of advantages. First, as Feridex® is known to tightly complex with PLL through electrostatic interactions, retention of Feridex® within the alginate-poly-L-lysine-alginate microcapsule may be enhanced. Furthermore, as Feridex® is available as a purified, stable, dextran-coated particle in a liquid suspension, the formation of iron aggregates within capsules is unlikely. This lack of aggregation may give magnetocapsules superior mechanical strength as compared to the magnetized capsules that use the paramagnetic or superparamagnetic metal to crosslink the alginate polymer. Finally, as Feridex® is an FDA-approved ferumoxide formulation, its safety is well established.

Magnetocapsules, according to some embodiments of the invention, may be desirable because they a) can be synthesized using clinically used and clinically approved materials; b) will not be subject to dilution by cell division or dislodging of labeled macrophages/stromal cells from islets in vivo; c) may bypass potential label toxicity issues; d) should not inhibit insulin secretion as opposed to direct intracellular labeling; and e) can provide potential information on capsule rupture and exposure of naked islets to an immunohostile environment.

In some embodiments, methods are provided of synthesizing an MRI-detectable microcapsule, comprising forming a droplet comprising a cell, an alginate polymer that is not crosslinked with a paramagnetic or superparamagnetic metal, and at least one of a paramagnetic or a superparamagnetic metal; adding a crosslinking agent to crosslink the alginate polymer; introducing the crosslinked droplet to a polycationic polymer solution; and introducing the polycationic polymer-treated crosslinked droplet to an alginate polymer solution. According to some embodiments of the invention, provided are methods of synthesizing a radiopaque microcapsule, or X-caps, comprising forming a droplet comprising a cell, an alginate polymer and a radiopaque contrast agent; adding a crosslinking agent to crosslink the alginate polymer; introducing the droplet to a polycationic polymer solution; and introducing the droplet to an alginate polymer solution. The crosslinking agent may be any agent known in the art for crosslinking alginates, such as Ca²⁺, Ba²⁺, Mg²⁺, Fe²⁺, Mn²⁺ and any combination thereof, and the like. U.S. Pat. Nos. 5,916,790 and 6,465,226, which contents are incorporated herein by reference in their entirety, cite further divalent cations that may be used.

In additional embodiments, the present invention provides a microcapsule for implantation into a mammalian body, comprising: a) at least one cell and/or biological agent; and b) a biocompatible semi-permeable alginate layer encapsulating the at least one cell, wherein the biocompatible semi-permeable alginate layer comprises a paramagnetic or superparamagnetic metal that does not participate in the crosslinking of the alginate layer. The paramagnetic or superparamagnetic metal can be iron, gadolinium, manganese, dysprosium and any combination thereof. In particular embodiments, the paramagnetic or superparamagnetic metal can be iron, which can be present in the microcapsule as a ferum-oxide. In certain embodiments, the ferum-oxide is derived from a Feridex® or Resovist® aqueous colloidal solution.

A method of synthesis of this microcapsule is also provided comprising: a) forming a droplet comprising: a cell and/or biological agent, an alginate polymer that is not crosslinked with a paramagnetic or superparamagnetic metal, and at least one of a paramagnetic or a superparamagnetic metal; and b) adding a crosslinking agent to crosslink the alginate polymer. In some embodiments, the crosslinking agent can be a divalent metal cation, which can be, for example, Ca²⁺, Ba²⁺, Mg²⁺, Fe²⁺, Mn²⁺ and any combination thereof. In certain embodiments, the alginate polymer solution does not comprise a paramagnetic or superparamagnetic metal.

The present invention further provides a method of embolizing a vascular site through physical obstruction, comprising introducing into the vascular site one or more microcapsules comprising a biocompatible semi-permeable membrane, wherein the biocompatible semi-permeable membrane comprises: at least one polycationic polymer region, at least one alginate polymer region, and a paramagnetic or superparamagnetic metal that does not participate in the crosslinking of the alginate polymer.

Introduction of a microcapsule of this invention for embolization can be carried out according to delivery protocols as described herein and as are well known in the art. The ability to identify the microcapsule by MRI, X-ray and/or ultrasound according to the methods of this invention allows for localization of the microcapsule to a target site for embolization as well as to identify and/or diagnose a vascular site that is partially or completely occluded.

The microcapsules and compositions of this invention can also be used for embolization, for example, to inhibit blood flow for a therapeutic effect, e.g., uterine fibroid embolization to inhibit circulation to and/or from a uterine fibroid, or tumor embolization to inhibit circulation to and/or from a tumor.

Nonlimiting examples of vascular sites of this invention include an aneurysm (e.g., vascular aneurysm, intracranial aneurysm, anterior circulation aneurysm, posterior circulation aneurysm), an artery, a vein, a lymph duct, a fistula, an arteriovenous malformation, a telangiectasia and the like, as would be known to one of ordinary skill in the art.

As used herein, a radiopaque contrast agent is one that renders the microcapsule detectable using X-ray radiological methods, including fluoroscopy and computed tomography. Examples of radiopaque contrast agents include radiopaque bismuth or barium compounds, such as barium sulfate and bismuth sulfate, and stabilized complexes containing Bi or Ba: Iodine containing compounds, such as 2,3,5 Triiodobenzoic acid, 3,5-Diacetamido-2,4,6-triiodobenzoic acid (Hypaque), 5-(acetyl-(2,3 dihydroxypropyl)amino)-N,N′-bis(2,3-dihydroxypropyl)-2,4,6-tri iodo-benzene-1,3-dicarboxamide (iohexyl), etc., can also be added to the microcapsules. Tantalum and tungsten compounds may also be used. Combinations of radiopaque contrast agents may also be used. Furthermore, radiopaque contrast agents may be used in microcapsules in combination with the paramagnetic and/or superparamagnetic metals described above. Thus, in some embodiments of the invention, the microcapsules can be both radiopaque and detectable by MRI (“XNMR-caps”).

For example, in a specific embodiment, islet cells are first suspended in a solution of 2% w/v ultrapurified sodium Protanal HF® alginate with 5% weight/volume 2,3,5 triiodobenzoic acid, 5% weight/volume bismuth sulfate (Sigma, St. Louis, Mo.) or 5% weight/volume barium sulfate (Sigma, St. Louis, Mo.) added. Spherical droplets are formed by the electrostatic interaction coupled with syringe pump extrusion and are collected in a 100 mM calcium chloride solution. The gelled droplets are suspended in 0.05% poly-L-lysine (Sigma, molecular mass=22-24 kDa). The droplets are washed with 0.9% saline and resuspended in 0.15% Keltone HVCR alginate for 5 min. Capsules are then washed with 0.9% saline.

In some embodiments, a radiopaque contrast agent, such as iodine, may be crosslinked directly to alginate. This technique obviates the need for utilizing an organic agent to incorporate iodine into the microcapsule. In addition, this approach may limit the leaching of the contrast agent from the microcapsule. An exemplary method of forming an iodine-crosslinked alginate is as follows. First, the alginate is neutralized with lithium hydroxide. Periodic acid (H₅I0₄) is then added to form a reaction (via nucleophilic addition of the alcohol group on periodic acid) with the carboxylic acids on the guluronate and mannuroate chains in alginate. Once stably formed, the mixture can then be brought back to an appropriate pH, such as pH 7.4, resulting in an iodine crosslinked alginate.

In some embodiments, the radiopaque contrast agent is also detectable by magnetic resonance imaging and/or by ultrasonography. For example, in some embodiments, the radiopaque contrast agent may be a perfluorocarbon (PFC). A perfluorocarbon refers to a hydrocarbon compound wherein most or all of the hydrogen atoms have been substituted with fluorine atoms. Exemplary PFCs include brominated PFC such as perfluorooctylbromide (PFOB) and perfluoropolyethers (PFPE) such as perfluoro (crown ethers). PFOB (C₈F₁₇Br) is a linear molecule containing a residual bromine atom that has significant radiopacity to be detected under CT. PFPE is crown ether that is particularly attractive as a MR imaging agent as all fluorine atoms are spectroscopically equivalent. While both are suitable MRI contrast agents, PFOB microcapsules display trimodal imaging capabilities and are detectable under ¹⁹F MRI, CT, and US.

In some embodiments, the perfluorocarbon is also detectable by magnetic resonance and by ultrasonography.

The incorporation of PFCs into microcapsules is attractive for a number of reasons. By exploiting the various features of PFCs, fluorinated biomaterials can be used to create smart scaffolds capable of providing information on perfusion of the encapsulated graft by monitoring O₂ tension noninvasively with MRI. In addition to providing a means of assessing pO₂. PFCs can also increase local oxygen tension. The ability to increase oxygen availability is paramount for the advancement of encapsulation therapy as many studies have suggested that graft failure occurs due to the lack of direct vascularization of the enclosed cells. This results in gradual tissue necrosis and death of encapsulated cells. In some embodiments, in vivo applications of these capsules with perfluorocarbon reservoirs could “reload” themselves by picking up O₂ from plasma that perfuses through the matrix. By acting as oxygen sinks, PFC containing microcapsules may have broad implications for increasing the viability of many encapsulated cell types.

Emulsions suitable for use in the microcapsule preparations of this invention may be prepared, for example, by adding two parts by volume of a brominated perfluorocarbon to 1 part by volume of lactated Ringer's solution containing a small amount (e.g., 6%) of an emulsifing agent, e.g., Pluronic F-68, and agitating on a vortex or sonicator until a stable emulsion is formed. More concentrated emulsions are formed by adding neat perfluorocarbon, up to a ratio of 12:1 by volume, and mixing until a stable emulsion is formed. Concentrated emulsions of this type, particularly those having perfluorocarbon/aqueous phase ratios of 6:1 to 10:1, will most likely be most useful for this microcapsule approach.

In addition to increasing local oxygen concentrations, there is some evidence that PFCs may have the additional advantage of enhancing the immunoisolatory properties of alginate microcapsules by acting in an immunomodulatory manner. Thus, PFC loaded alginate capsules could further reduce rejection of cellular therapeutics in immunocompetent hosts.

A final potential advantage of incorporating PFCs in microcapsules is that it provides a means of tracking cells using X-ray imaging modalities, MRI or ultrasound. X-ray and ultrasound guided procedures are the preferred method for minimally invasive interventions at present. For this reason, PFC microcapsules could prove an ideal vehicle for targeted delivery of cellular agents. Further, as fluorocapsules are detectable with MRI, follow-up examinations with MRI may be performed while avoiding radiation exposure.

Like radionuclide tracers, there is essentially no endogenous fluorine signal in vivo. Thus, ¹⁹F “hotspot” MRI can be performed for tracking of the microcapsules. In phantom studies using a high field scanner (e.g., 9.4 T), fluorocapsules may be detected. Since the first clinical 7 T MR scanners are currently being installed, it can be expected that ¹⁹F MRI will be possible in humans. However, the advantage of being able to deliver and visualize fluorocapsules alone is of great benefit, and the potential for MRI/MRS spectroscopy enhances enthusiasm for the PFC microcapsules.

As certain PFCs can be imaged with ultrasound (US), MRI and x-ray modalities, a final potential advantage of incorporating PFCs into microcapsules is the ability to non-invasively monitor capsule location. Such information could prove invaluable in determining fundamental questions such as ideal transplantation site and best means of delivery of such grafts. Although detectable under ¹H MRI, superparamagnetic iron oxides are not detected directly but instead are detected from a misalignment of the orientation of water protons, caused by microscopic disturbances of the magnetic field. PFC contrast agents take a different approach to molecular labeling than traditional contrast agents. Fluorinated contrast agents are detected directly by ¹⁹F MRI, assuring a lack of background signal as the body lacks any endogenous fluorine. As a result, when imaging fluorinated contrast agents, there is no uncertainty about the signal source. Furthermore, the fluorine signal offers a hotspot interpretation when superimposed on anatomical ¹H scans, which can be taken during the same session. Additionally, certain PFCs have significant radiopacity for visualization under X-ray imaging.

A means of assessing adequate perfusion by determining local oxygen saturation could prove invaluable for a better understanding of the long-term viability of encapsulated grafts after transplantation. In the case of PFCs, apolar oxygen imparts paramagnetic relaxation effects on ¹⁹F nuclei associated with spin-lattice relaxation rates (R₁) and chemical shifts. This effect is proportional to the partial pressure of oxygen (pO₂). If incorporated into grafts containing encapsulated cells, PFCs in combination with ¹⁹F MRI could provide a non-invasive means of determining graft perfusion. Furthermore, as superimposition of CT and MRI scans, using hybrid X-Ray/MR imaging systems, becomes more frequent, capsules with multimodal contrast agents, such as PFOB, will allow researchers and clinicians to accurately monitor encapsulated cells in vivo.

Also provided are compositions comprising a microcapsule described herein, in a pharmaceutically acceptable carrier. The term “pharmaceutically acceptable carrier” is used herein and in the claims to refer to a carrier medium that does not significantly alter the biological activity of the active ingredient (e.g., the antiviral activity of a compound according to the present invention) to which it is added. The one or more substances of which the pharmaceutically acceptable carrier is comprised, typically depend on factors (or desired features for its intended use) of the pharmaceutical composition such as the intended mode of administration, desired physical state (e.g., solid, liquid, gel, suspension, etc.), desired consistency, desired appearance, desired taste (if any), desired pharmacokinetic properties once administered (e.g., solubility, stability, biological half life), desired release characteristics (e.g., (a) immediate release (e.g., fast-dissolving, fast-disintegrating), or (b) modified release (e.g., delayed release, sustained release, controlled release)), and the like. As known to those skilled in the art, a suitable pharmaceutically acceptable carrier is typically sterile and may comprise one or more substances, including but not limited to, a diluent, water, buffered water, saline, 0.3% glycine, aqueous alcohol, isotonic aqueous buffer; a water-soluble polymer, glycerol, polyethylene glycol, glycerin, oil, salt (e.g., such as sodium, potassium, magnesium and ammonium), phosphonate, carbonate ester, fatty acid, saccharide, polysaccharide, stabilizing agent (e.g., glycoprotein, and the like for imparting enhanced stability, as necessary and suitable for manufacture and/or distribution of the pharmaceutical composition), excipient, preservative (e.g., to increase shelf-life, as necessary and suitable for manufacture and distribution of the pharmaceutical composition), bulking agent (e.g., microcrystalline cellulose, and the like), suspending agent (e.g., alginic acid, sodium alginate, and the like), viscosity enhancer (e.g., methylcellulose), taste enhancer (e.g., sweetener, flavoring agent, taste-masking agent), binder (generally, to impart cohesive quality to a tablet or solid formulation; e.g., gelatin, natural and/or synthetic gums, polyvinylpyrrolidone, polyethylene glycol, and the like), extender, disintegrant (e.g., sodium starch glycolate, sodium carboxymethyl cellulose, starch, and the like), dispersant, coating (generally to impart a surface active agent to a tablet or solid formulation; e.g., polysorbate, talc, silicon dioxide, and the like), lubricant (e.g., magnesium stearate, calcium stearate, sodium lauryl sulphate, and the like), or colorant.

Furthermore, methods of delivering a cell to a mammal (e.g., a human) comprising introducing the microcapsule according to an embodiment of the invention into the mammal, are provided herein. In some embodiments, the microcapsule is introduced by injecting the microcapsule into the mammal via a magnetic resonance-detectable needle. In addition, in some embodiments, the microcapsule is injected into the mammal, e.g., into the portal vein of the mammal, in a pharmaceutically acceptable carrier. Various methods of delivering cells to animal are well known in the art. In further embodiments as described herein, the microcapsules of this invention can be used as embolic agents and their detection by MRI, X-ray and/or ultrasound enables verification of successful embolization.

The present invention further provides microcapsules that comprise various biological or bioactive agents, such as drugs, factors, and/or other cytokines that may be included within the capsules either with or without cells of this invention. Thus, in some embodiments, the biological or bioactive agent can be present in the microcapsule in the absence of any cells in the microcapsule. In further embodiments, the microcapsules of this invention can comprise cells that are genetically engineered to produce various bioactive agents, such as, for example, cytosine deaminase [as an example of an enzyme that converts a prodrug to a toxic chemotherapeutic (5-fluorocytosine to 5-fluorouracil), thereby sparing the encapsulated cell but making the environment near the tumor toxic], angiostatin, inhibiting factors for tumors etc, as well as enhancing/stimulating factors such as cytokines that stimulate immune cells to fight cancer (e.g., interferon beta, interferon gamma, interleukins etc). These bioactive agents and/or cells and/or genetically engineered cells can be present in any combination in the microcapsules of this invention.

### Imaging of the Microencapsulated Cells

According to some embodiments, provided herein are apparatuses for imaging microcapsules according to embodiments of the invention. The apparatus may include X-ray systems (e.g., computed tomography (CT) systems, digital X-ray systems, and the like), ultrasound systems, and magnetic resonance imaging (MRI) systems. In some embodiments, the microencapsulated cells can be tri-modal and can be visualized using all three types of imaging systems.

In some embodiments, an MRI system for MRI imaging of a microcapsule according to an embodiment of the invention can include an MRI scanner, and associated circuits, including, for example, an RF amplifier, a gradient amplifier and a receiver configured to detect the magnetic resonance signal produced from the microcapsule(s). The MRI scanner can also be in communication with a display for substantial real-time tracking for MR-guided interventional procedures. Thus, real-time delivery and tracking of microcapsules according to embodiments of the invention may be achieved with clinical MRI scanners. The MRI scanners can include magnets having any suitable magnetic field strength (B₀). Conventional imaging magnets are 1.5 T superconducting magnets, however, lower, and typically, higher field strength magnets may be used. For example, 2 T, 3 T, 6 T, 9 T or even greater field strength magnets may be used. Examples of MRI systems include, but are not limited to, those provided by General Electric Medical Systems, Siemens, Philips, Varian, Bruker, Marconi, Hitachi and Toshiba.

To surgically perform islet cell transplantation, proper imaging strategies dedicated to the portal venous system can be used. MR protocols, as well as modifications of the MR-active probes and/or needles have been developed, so that MR-guided punctures of the portal vein are feasible and safe using a clinical MRI scanner. See, e.g., Arepally et al., Radiology, 2006, 238, 113-118. The term “MRI-active” refers to a delivery device (typically a needle) that is visible in MRI images. The MRI active devices may be used to guide placement of the cells, needle and/or interventional delivery device and are not necessarily used to generate images of local structure. The MRI active device may function as a receive antenna to detect local MRI signals. For disclosures of exemplary MRI active devices, see, e.g., U.S. Patent Publication Nos. 2003/0028094; 2003/0050557; and U.S. Pat. Nos. 5,928,145 and 6,701,176, the entire contents of these documents being incorporated herein by reference as if recited in full herein. The delivery device 150 can comprise a tube or catheter and may, as shown, include a needle to precisely introduce the target cells. The delivery device can include an MRI receiver antenna that can be a loopless antenna, a whip antenna, a coil antenna, and/or a looped antenna. See, e.g., U.S. Pat. Nos. 5,699,801; 5,928,145; 6,263,229; 6,606,513; 6,628,980; 6,284,971; and 6,675,033, the contents of which are incorporated by reference herein as if recited in full herein.

Since pertinent vessels, such as the inferior vena cava (IVC) and the portal vein, are in close proximity to vital organs, any attempt to puncture the target vessel will typically require a very precise and direct path. Thus, a safe puncture of the portal vein through a transcaval approach typically employs substantially real-time visualization of all pertinent structures and the ability to perform multiplanar and 3D projections in order to more precisely navigate the path of the needle to the target structure.

One exemplary technique is to access the portal vein through a transfemoral IVC approach rather than via a transhepatic approach. In this procedure, accessing the portal vein is achieved by performing a vascular puncture from the IVC. By approaching the portal vein from this transcaval approach, several advantages are provided. The retroperitoneum provides a safe space that is capable of providing a seal to vascular punctures; as has been well-demonstrated for decades with transcaval aortography and transcaval placement of venous catheters. By providing a natural seal around these vessels, a retroperitoneal approach to the portal vein allows for repeated safe access into the mesenteric system. In addition, accessing the portal vein from a transcaval approach under MRI allows for easy navigation to either the right or left portal vein, with the added benefit of performing high resolution imaging of the liver with intravascular MR guidewires.

Utilizing both the MR-active/visible needle (See Arapally et al., J Magn Reson Imaging, 2005, 21, 463-467; Karmarkar et al. “Trackable Intramyocardial Injection Catheter” Magnetic Resonance in Medicine, 2005) and a real-time slice navigation (iDrive; GE Healthcare, Milwaukee, Wis.) interface, this technique allows for reliable and safe access to the portal vein in a MR environment. Real-time slice navigation using a steady-state, free precession sequence allows for rapid data acquisition with direct manipulation of slice prescription, flip angle, slice thickness, and field of view, all in a real-time setting. Using this sequence, the vessel and vasculature can be visualized with high contrast to surrounding soft tissues without the need for gadolinium-based contrast agents. This real-time sequence for the portal vein may be desirable because (1) due to the T2/T1 effects on blood of the steady state sequences, all vessels may be adequately visualized in an axial plane; (2) rapid multiplanar capabilities may allow the punctures to be monitored; and (3) adequate temporal resolution (8-10 frames/sec) to perform real-time manipulation of the needle may be realized. The use of this system has provided a significant improvement in obtaining multiplanar views of the vasculature, needle, and target organs, such as the liver. In all cases, the needle can be fully visualized as it traverses the retroperitoneum and enters the mesenteric vein or portal vein. In the vast majority of cases (90%), access into the portal circulation has been achieved with one puncture, without complications. MR imaging also confirmed that the needle did not traverse any retroperitoneal organs or vessels.

One advantageous aspect of accessing the portal system under MR guidance is the opportunity to not only deliver therapeutic agents to the pancreas and liver but also to perform high-resolution imaging of these target organs. More specifically, using this access can allow for microimaging of microcapsules according to embodiments of the invention in an animal model to evaluate engraftment or destruction.

However, as noted above, other imaging modalities may be used to image the microcapsules in vivo. Thus, according to other embodiments of the invention, apparatuses for imaging microcapsules according to the present invention comprise an X-ray source, and an X-ray detection device and output circuit that generates visual data associated with the location of the microcapsules (and delivery device) in a position in the body. X-ray techniques are well known to those of skill in the art.

In the methods of the present invention, the microcapsules and compositions of this invention can be introduced or delivered to a subject according to various protocols for administration, including but not limited to, intravenous, intraarterial, intramuscular, intracardiac, intraperitoneal, intrapleural, subcutaneous, intracerebral, intrathecal, oral, nasal, respiratory and/or intradermal administration, including any combination thereof.

The present invention will now be described in more detail with reference to the following examples. However, these examples are given for the purpose of illustration only and are not to be construed as limiting the scope of the invention.

### EXAMPLES

### Example 1

Magnetocapsules (MR-CAPs) were synthesized by modifying the classic alginate/poly-L-lysine (PLL)/alginate (APA) microencapsulation protocol developed by Lim and Sun (Science, 1980, 210 (4472) 908-10), in which the PLL is used as a polycationic stabilizer for the microcapsules. In standard APA microcapsules, the positively charged amino group of the lysine molecule interacts with the negatively charged carboxyl and hydroxyl groups of the uronic acid (basic unit of alginate). The Lim and Sun synthesis procedure was modified by adding ferumoxides to the core layer of alginate that surrounds the islet.

After a typical 2-hour incubation period with 200 μg Fe per ml, MR-CAPs were found to have an iron content of 1.82±0.3 ng of Fe per capsule. This is about 2 orders of magnitude higher than the typical contents of Feridex®-labeled cells, which varies between 10-20 pg iron per cell. Magnetocapsules prepared with 200 μg Fe per ml were used throughout subsequent experiments. In contrast to human islets encapsulated without the Feridex® synthesis step, MR-CAP human islets exhibited a characteristic Feridex®-like color. Anti-dextran immunostaining (using a monoclonal antibody directed against the dextran coat of Feridex®) demonstrated that Feridex® labeling was quite uniform and not in the form of small aggregates that is typical for direct cell labeling. In vitro comparison of unlabeled APA microcapsules, MR-CAP βTC-6 murine insulinoma cells, and MR-CAP cadaveric human islets did not reveal any appreciable difference in permeability of capsules, islet viability, and insulin secretory response of encapsulated cells. Both unlabeled microcapsules and MR-CAPs were found to be permeable to lectins ≦75 kD, but were impermeable to lectins ≧120 kD (Table 1), thus ensuring the blockage of the penetration of antibodies while allowing free diffusion of insulin (˜5 kD) and nutrients.

The murine βTC-6 insulinoma cell line was magnetoencapsulated to assess the viability of MR-CAP cells in culture. A microfluorometric assay was performed to label all cells with Newport Green and dead cells with Propidium Iodide. This revealed that the viability of cells was 94% and 82% at 3 and 6 weeks of culture, respectively. These values did not differ from that of βTC6 cells encapsulated in unlabeled capsules (96% and 81% at 3 and 6 weeks, respectively).

TABLE 1
Permeability of non-labeled capsules and MR-CAPs for lectins with
various molecular weights.

| | Capsules − Feridex ® | Capsules + Feridex ® |
|---|---|---|
| 36 kD | ✓ | ✓ |
| 75 kD | ✓ | ✓ |
| 120 kD | X | X |
| 150 kD | X | X |

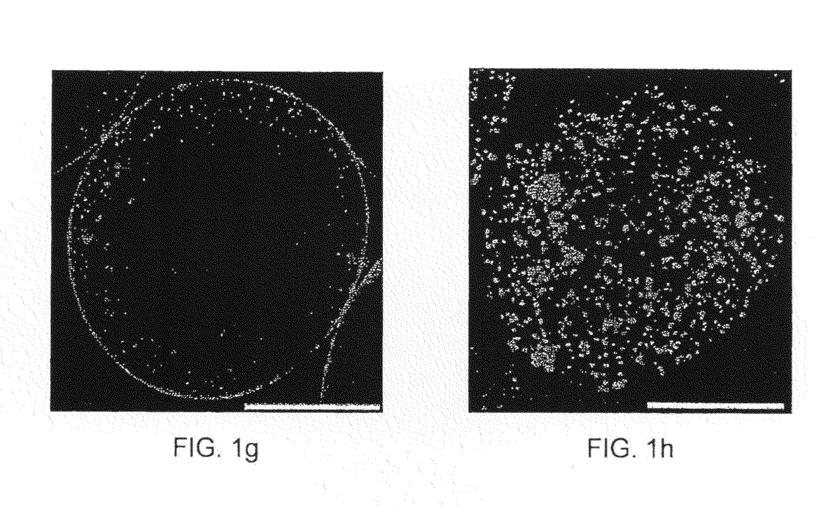

The insulin secretory response of MR-CAP human islets was compared against islets encapsulated without Feridex® over a 15-day period (FIG. 2). For each time point, a sample of cultured islet capsules was removed, and islets were counted and transferred into a well with fresh medium containing 4 mM glucose. Following incubation for 90 min, the total human insulin secretion was determined using an ELISA kit. Using the FDA-approved test for bioequivalence (TOST), with a threshold value of 5% and α=0.05, the insulin secretion from MR-CAP islets was compared to islets encapsulated without Feridex®. Except for a small decrease at day 3, the MR-CAP islet insulin secretion was found to be bioequivalent to secretion from islets encapsulated in unlabeled microcapsules, ranging between 2-2.5 ng insulin per islet (FIG. 2). This indicates that the addition of Feridex® to the microcapsules does not interfere with the “porosity” of the capsules and allows unimpeded diffusion of insulin across the capsule membrane.

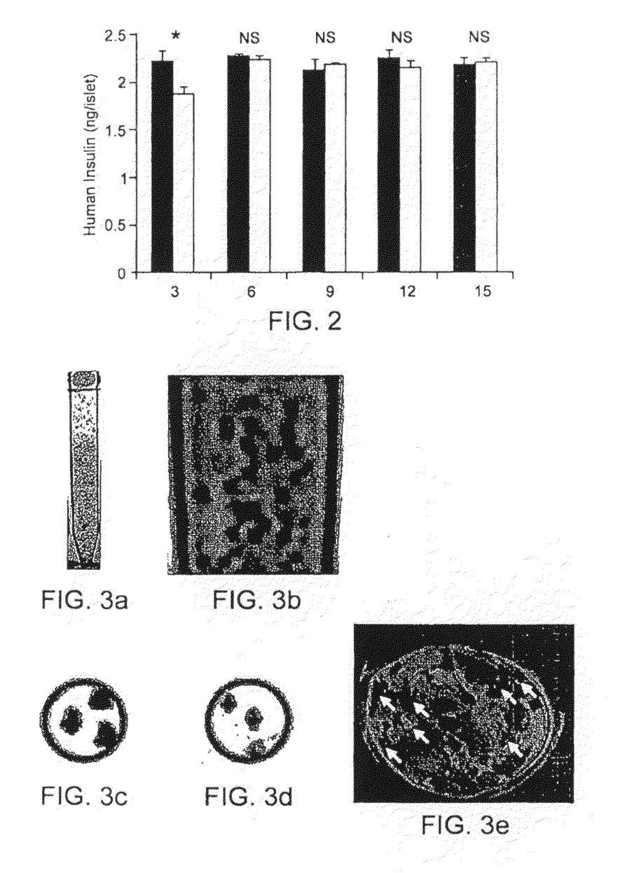

The MR detectability of MR-CAPs was also investigated. FIG. 3 shows that an incorporated Feridex® content of 1.8 ng Fe per capsule is sufficiently high to enable easy detection of single capsules both in agarose phantoms (FIGS. 3a-d) and in mice (FIG. 3e).

To evaluate the functionality of MR-CAP βTC6 murine insulinoma cells in vivo, 6,000 MR-CAPs, each containing 500 beta cells, for a total of 3×10⁶ cells (which is the approximate number of β-cells in 2,000 human islets), were transplanted into the peritoneal cavity of STZ-induced diabetic mice. Examination of blood glucose levels revealed that glucose levels normalized to about ˜100 mg/dl (for n=15) by 1 week of transplantation and remained constant throughout a period of 8 weeks. In contrast, non-transplanted animals remained hyperglycemic. MR-CAP cell-transplanted animals showed an increasing net weight gain during this period, while untreated animals lost a significant amount of weight. Compared to pre-transplantation levels, murine C-peptide levels were significantly increased at 4 weeks and 8 weeks post MR-CAP cell engraftment, in contrast to the control animals.

### Example 2

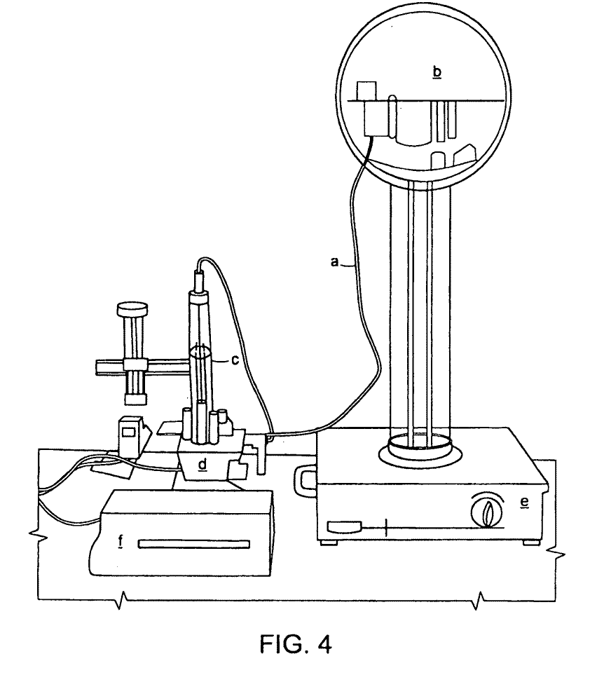

This synthesis of MR-CAPs is based on a modification of the original alginate capsule method of Lim and Sun. This modification involves the use of an electrostatic droplet generator, which produces smaller, stronger, and more uniform capsules compared to the older air-jet technique. The laboratory set-up is shown in FIG. 4. An ignition wire (FIG. 4a) is connected to the van de Graaff dome (FIG. 4b). The other end is connected to a 20 g 1½″ blunt needle. Human cadaveric islets are cultured in CMRL 1066 medium supplemented with 10% fetal calf serum, 1% penicillin/streptomycin, and 1 mM L-glutamine, using a humidified CO₂ incubator at 37° C. and a 5% CO₂ atmosphere. Before encapsulation, human cadaveric islets are first passed through a 20 g needle to remove large aggregates and impurities. The concentration of islet cells is adjusted to 400 islet equivalents/ml (about 5% of total volume when islets are settled). The needle is fitted on a I cc tuberculin syringe (FIG. 4c), which contains the islet cells suspended in 2% w/v ultrapurified sodium Protanal® HF alginate from FMC Biopolymers (Haugesund, Norway) with 20% vol/vol Feridex® (Berlex Laboratories, Inc., Wayne, N.J., stock=11.2 mg Fe/ml). A Petri dish (FIG. 4d), containing isotonic (1.70%) calcium chloride dihydrate, buffered with 10 mM HEPES, is placed under the needle. A stainless wire is immersed in the calcium solution and connected to a ground. The current is adjusted by changing the van de Graaff belt speed (FIG. 4e). The islets/alginate solution is passed through the needle with a flow rate of about 200 μl/min using a nanoinjector pump (FIG. 4f). Collected droplets, representing islet cells surrounded by the first layer of alginate, are washed three times in saline. After extrusion of alginate/Feridex/islet microspheres, they are collected in a solution of 100 mM CaCl₂ that complexes with the alginate to form stable capsules. These gelled droplets are suspended in 0.05% poly-L-lysine (Sigma, molecular mass=22-24 kDa) for 5 min. The droplets are washed with 0.9% saline and resuspended in 0.15% Keltone HVCR alginate (Monsanto, St. Louis, Mo.) for 5 min. The final step is washing with 0.9% saline. The rationale for using two different alginates is the relative ratios of mannuronate and guluronate (inner layer guluronate alginate has superior strength, while outer layer mannuronate alginate is less immunogenic).

The protocol above was successful for preparing MR-CAPs with 20% v/v Feridex® but the optimal iron loading of the capsules may vary. Excessively large amounts of iron may cause such an extensive bloom artifact on T2*-weighted MRI that the ability to detect single capsules is lost. On average, this protocol resulted in the encapsulation of 1 islet per capsule.

### Example 3

To set up the generator, a wire is attached (preferably an automobile ignition wire or the like) to a van de Graaff dome. The other end of the wire is connected to a 20 g 1½″ blunt needle. The needle is fitted on a 1 cc tuberculin syringe which contains the islets suspended in 0.8% high guluronate rich alginate in saline with 0.5 mM sodium citrate and 10 mM HEPES, pH 7. Concentration of islets is about 1-5%. The islets may need to be pre-screened to get rid of any clumps or large particles that can clog the 20 g needle.

A flow rate of about 200 microliters per minute may produce very small droplets. The process is very sensitive to viscosity and gelling properties of the alginate.

Before encapsulation, human cadaveric islets were first passed through a 20 g needle to remove large aggregates and impurities. The concentration of islet cells was adjusted to 400 islet equivalents/ml and that of βTC-6 to 1.5×10⁷ cells/ml. Cells were suspended in 2% w/v ultrapurified sodium Protanal® HF alginate (FMC Biopolymers) and 20% vol/vol Feridex® (Berlex Laboratories, stock=11.2 mg Fe/ml). This solution was passed through a needle at a flow rate of about 200 μl/min using a nanoinjector pump. Droplets, representing islet cells surrounded by the first layer of alginate, were collected in a Petri dish containing 100 mM CaCl₂, buffered with 10 mM HEPES, and then washed three times in saline. The gelled droplets were then suspended in 0.05% poly-L-lysine (Sigma, Mw=22-24 kDa) for 5 min to cross-link the alginate and Feridex®. The droplets were washed with 0.9% saline and resuspended in 0.15% Keltone HVCR alginate (Monsanto) for 5 min, and then finally washed with 0.9% saline.

### Example 4

### Exemplary Method of Producing a High Guluronate Alginate

Dissolve Protanal-HF alginate to 0.1% in 0.5 mM EDTA, 10 mM HEPES, pH 7.0. Filter to 0.45 microns to remove particulates. In a separate flask, bleach 4 gm fine mesh activated charcoal per gram alginate by resuspension to 4% (w/v) in 0.1 M sodium perchlorate. After 30 minutes mixing, wash the bleached charcoal by centrifugation (5 min @ 500×g) twice with water, 4× with ethanol, 4× with water. Discard the supernatants. Add the bleached activated charcoal slurry to the filtered alginate and stir for 30 min to adsorb organic contaminants. Filter the supernatant to 0.22 micron; then filter at 0.1 micron. Add 10.2 ml 10% MgCl₂ 5H₂O per liter filtrate and mix thoroughly.

Gradually add 3.8 ml 34% CaCl₂-2H₂O while stirring and mix for 30 min to precipitate the higher molecular weight, guluronate-rich chains. Spin 20 min at 2,000×g. Discard supernatant. Estimate pellet volume and add 2 volumes 0.1 M EDTA, 10 mM HEPES, pH 7.0. Q.s. H₂O to 500 ml per gram alginate starting material. Adjust pH to 7.0 if necessary. Concentrate 10 fold by ultrafiltration to 10 kD to remove small fragments.

Dilute retentate to starting volume with water and reconcentrate. Repeat, if necessary.

Dilute retentate back to starting volume again with water. Add 1/20 volume 2.5 M NaCl.

While vigorously stirring, slowly add an equal volume of ethanol. Spin 10 min at 500×g. Discard supernatant. Redissolve in 120 mM NaCl, 0.5 mM EDTA (200 ml per gram alginate starting material). While vigorously stirring, slowly add 4 volumes of ethanol.

Spin 10 min at 500×g. Discard supernatant. Thoroughly resuspend pellet in 1 liter ethanol per gram alginate starting material (will not dissolve). Spin 10 min at 500×g. Discard supernatant. Thoroughly resuspend pellet again in 1 liter ethanol per gram alginate starting material. Collect precipitate on fine mesh stainless steel sieve. Press out excess liquid. Tease with forceps to fluff precipitate. Dry at 60° C. in vacuo. The resulting alginate should be stored in a cool, dry place until ready for use. Dissolve to desired concentration in buffer (e.g., 10 mM HEPES buffered normal saline with 0.5 mM sodium citrate) and sterilize by filtration.

### Example 5

### Exemplary Method of Producing a High Mannuronate Alginate

Dissolve Keltone HVCR (by Monsanto) alginate to 0.1% in 0.5 mM EDTA, 10 mM BEPES, pH 7.0. Filter to 0.45 microns to remove particulates. In a separate flask, bleach 4 gm fine mesh activated charcoal per gram alginate by resuspension to 4% (w/v) in 0.1 M sodium perchlorate. After 30 minutes mixing, wash the bleached charcoal by centrifugation (5 min @ 500×g) twice with water, 4× with ethanol, 4× with water. Discard the supernatants. Add the bleached activated charcoal slurry to the filtered alginate and stir for 30 min to adsorb organic contaminants. Filter the supernatant to 0.22 micron, then 0.1 micron. Concentrate 10 fold by ultrafiltration to 10 kD to remove small fragments. Dilute retentate to starting volume with water and reconcentrate 10 fold. Repeat, if necessary. Dilute retentate back to starting volume again with water. Add 1/20 volume 2.5 M NaCl. While vigorously stirring, slowly add an equal volume of ethanol. Spin 10 min at 500×g. Discard supernatant. Redissolve in 120 mM NaCl, (100 ml per gram starting material). Repeat last three steps. While vigorously stirring, slowly add 4 volumes of ethanol. Spin 10 min at 500×g. Discard supernatant. Thoroughly resuspend pellet in 1 liter ethanol per gram alginate starting material (will not dissolve). Spin 10 min at 500×g. Discard supernatant. Thoroughly resuspend pellet again in 1 liter ethanol per gram alginate starting material. Collect precipitate on fine mesh stainless steel sieve. Press out excess liquid. Tease with forceps to fluff precipitate. Dry at 60° C. in vacuo. Store the dry purified alginate in a cool, dry place until ready for use. Dissolve to desired concentration in buffer (e.g., 10 mM HEPES buffered normal saline with 0.5 mM sodium citrate). Sterilize by filtration.

### Example 6

An MR-guided transplantation of MR-CAP human islets was performed in a swine model on a 1.5 T clinical MR scanner. In this animal model, human islets were procured from the Islet Cell Resource Center, magnetoencapsulated using a method as described herein and transplanted into a swine using MR fluoroscopy with follow-up MRI and monitoring for 3 weeks.

The transplantation procedure was performed completely under MRN on a clinical 1.5 T (CVi, GE, Milwaukee, Wis.) system. Under general anesthesia, a standard clinical 12 F sheath was placed in the common femoral vein and the MR-trackable needle was introduced as previously described. Under a real-time steady state free precession (SSFP) sequence with multiplanar views, the needle system was then guided through the IVC and into the portal vein. Once the needle had entered the portal vein, a slow infusion of 40,000 MR-CAPs with real-time monitoring was performed. Following delivery, MR-CAP distribution was assessed using conventional receiver coils with a gradient echo pulse sequence, T2* (TR/TE: 3.5/1.2 ms, flip angle: 45°. Following transplantation of MCs, animals were closely monitored for 3 weeks. Human C-peptide could be detected for the three weeks follow-up. During this period, no apparent health complications occurred. Post-transplantation, MR scanning on a clinical 1.5 T MR scanner revealed grafting of MR-caps throughout the entire liver. As a result of MR-cap human islet grafting, specific human C-peptide could be detected for the three weeks in which the swine survived. Histopathology confirmed the MRI findings of global liver engraftment.

### Example 7

Human islets isolated from a brain-dead donor were provided by the Joslin Diabetes Research Center (Boston, Mass.) under an approved protocol of the Islet Cell Resource Center and were cultured in RPMI 1640 medium (Gibco) supplemented with 10% fetal calf serum, 1% penicillin/streptomycin/L-glutamine (all reagents from Sigma Co.) in a humidified CO₂ incubator at 37° C. and 5% CO₂ atmosphere. With microencapsulated cells, groups of 10 microcapsules, each initially containing ˜1 islet, were cultured in tissue culture multiwell plates. Culture medium was exchanged every three days.

Protanal HF alginate from FMC Biopolymers (Haugesund, Norway) and Keltone HVCR alginate from Monsanto (St. Louis, Mo.) was first purified with filtration through a 0.2 μm-pore-size filter in order to achieve necessary purification and sterility. Purified alginate was then utilized to microencapsulate human islets with an electrostatic droplet generator. Islet cells were first suspended in a solution of 2% w/v ultrapurified sodium Protanal HF alginate with 5% weight/volume 2,3,5 triiodobenzoic acid, 5% weight/volume bismuth sulfate (Sigma, St. Louis, Mo.) or 5% weight/volume barium sulfate (Sigma, St. Louis, Mo.) added. Spherical droplets were formed by the electrostatic interaction coupled with syringe pump extrusion and were collected in a 100 mM calcium chloride solution. The gelled droplets were suspended in 0.05% poly-L-lysine (Sigma, molecular mass=22-24 kDa). The droplets were washed with 0.9% saline and resuspended in 0.15% Keltone HVCR alginate for 5 min. Capsules were then washed with 0.9% saline.

Following magnetoencapsulation, viability of human islets was determined by a microfluorometric assay in which viable cells were labeled with Newport Green and dead cells with propidium iodide. To this end, encapsulated islet cells were incubated with 10 mM Newport Green (Sigma, St. Louis, Mo.) for 30 minutes and 5 mM propidium iodide (Sigma, St. Louis, Mo.) for 10 min. Newport Green was excited using the 500-nm laser line, and the emitted fluorescence was detected through a 535-nm long-pass filter. Propidium iodide was excited using the 514-nm laser line, and the emitted fluorescence was detected through a 550-nm long-pass filter. Cells fluorescing red were counted as dead and cells fluorescing green were counted as viable. In cases of dual partial red and green staining, cells were counted as dead. Seven representative microcapsules from each preparation were randomly selected for cell viability determinations. Random selection of microcapsules was accomplished by vortex mixing each tube just prior to micropipetting a 100-μL sample from the tube onto a glass slide. Viability at different time points was assessed for three separate microencapsulation procedures for each type of microcapsule.

In order to determine permeability of Ba X-Caps and Bi X-Caps as compared to APA controls, the various microcapsules were incubated with one of four fluorescently labeled lectins of varying molecular weight. Lectin incubation consisted of either incubation with 15 μL (1 mg/μL) of FITC-Triticum vulgare (WGA, MW: 36,000 kD), FITC-Maackia amurensis I (MAL-I, MW: 75,000 kD), FITC-Ricinus communis (RCA-I, MW: 120,000 kD) or FITC-Sambuca nigra (SNA, MW: 150,000 kD). All lectins were obtained from EY Lab Inc. except FITC-Maackia amurensis I (Vector Laboratories). Magnetocapsules were incubated for 48 h at 4° C. on a mechanical rocker, after which they were examined microscopically (Olympus X51 and IX71 epifluorescence microscopes equipped with an Olympus DP-70 digital acquisition system).

Capsules were embedded with Vectashield mounting medium (Vector, Burlingame, Calif., USA) and examined for fluorescence with the previously described epifluorescence microscope setup. Macroscopic images of microcapsules were obtained with a D100 6 MP Digital SLR Camera (Nikon; Melville, N.Y.).

A static incubation assay was used to assess the insulin secretion response of microencapsulated human islets. Microencapsulated islets were placed in a culture insert (membrane pore diameter 12 μm; Millicell PCF, Millipore, France). The insert was put into a well of a 24-well culture-plate (Falcon Multiwell; Becton, Dickinson). Insulin secretion was measured after 1.5 hrs in a solution of specific glucose level. Specifically a step-wise increase in glucose concentration from a 6 mM to a 7 mM to a 8 mM D-glucose concentration in RPMI 1640 medium was employed to assess the fine glucose responsiveness of encapsulated cells. Aliquots of the medium were stored at −80° C. The C-peptide content of the samples was determined with an enzyme linked immunosorbent assay (ultrasensitive human C-peptide ELISA, Alpco Diagnostics, Windham, N.H.); results (in ng/ml) were expressed as a means of three independent experiments. C-peptide secretion (ng/islet) from human islets encapsulated in the various capsule preparations during incubation in 8 mM glucose solution for 90 minutes was also assessed after 7 days and 14 days in culture

Female C57/BL mice (Charles River), age 6-8 weeks, were used as recipients for microcapsules. Before transplantation, mice were anesthetized with ketamine (65 mg/kg i.p.; Pfizer) and xylazine hydrochloride (13 mg/kg i.p.; Bayer). For all transplants, microcapsules were injected into the peritoneal cavity with a 20-gauge needle. The anesthetized mice were strapped in a supine position to a table and a total of 5,000 capsules was injected under fluoroscopic guidance.

Rabbits weighing 3 to 4 kg were preanesthetized with acepromazine (1 mg/kg) mixed with ketamine (40 mg/kg) IM. An intravenous catheter was placed in the ear vein and the rabbit was induced with thiopental (to effect ˜10 mg/kg). The rabbit was then intubated to maintain an open airway. General anesthesia was maintained with intravenous thiopental. The anesthetized rabbits were strapped in a supine position to a table and a total of 2,000 Ba X-Caps and 2,000 Bi X-Caps was injected intramuscularly under fluoroscopic guidance in the hind limb of the rabbit.

Results were expressed as means±S.E. Statistical analysis of the data was conducted by a one-way ANOVA, and significance was indicated by P<0.05. Data was also analyzed using bioequivalence (BE) testing using the Two-One Sided T-test approach (TOST).

The synthesis of radiopaque microcapsules is a modification of the classic alginate/poly-L-lysine/alginate (APA) microencapsulation protocol developed by Lim and Sun. In order to make microcapsules of diameters <350 μm, an electrostatic droplet generator was substituted for a traditional air-droplet generator to encapsulate human islet cells. Furthermore, the traditional synthesis of capsules was modified by adding contrast agents to the core layer of a high guluronate alginate that surrounds the islet. The outer layer of the APA capsule made with high mannuronate alginate was left contrast free in order to avoid any potential inflammatory reaction due to contrast on the capsule surface. The high guluronate alginate for the inner alginate layer in which the islet is contained was chosen for its relative strength. For the outer layer a high mannuronate alginate was chosen, as it has been shown to be less immunogenic. Immediately following the encapsulation process, islet recovery was 90+/−4% (n=14 independent encapsulation procedures). Total volume of encapsulated human islets was calculated in a 15 ml Falcon centrifuge tube. The mean volume of 1000 encapsulated and nonencapsulated IE was 40+/−3 μl and 3.6+/0.4 μl.

For an immunoisolation device to function properly, its permeability property is of critical importance. It was conceivable that the incorporation of the contrast agents may have changed the permeability of the microcapsules. The contrast agents might occlude some of the channel space available for solute diffusion, hence increasing the permeability threshold. Fortunately, this did not prove to be the case. Traditional APA microcapsules, Ba X-Caps, Bi X-Caps were found to have equal permeability to fluorescent lectins. Specifically, all capsule types were found to be permeable to fluorescent lectins <75 kD but were found to be impermeable to lectins >120 kD, thus blocking antibodies while allowing penetration of smaller nutrients and secretion of insulin (Table 2).

TABLE 2
Permeability of barium x-caps, bismuth x-caps and APA controls to
fluorescent lectins: FITC-Triticum vulgare (WGA, MW: 36 kD),
FITC-Maackia amurensis I (MAL-I, MW: 75 kD),
FITC-Ricinus communis (RCA-I, MW: 120 kD), or
FITC-Sambuca nigra (SNA, MW: 150 kD).

| | Barium | Bismuth | Control |
|---|---|---|---|
| 36 kD | ✓ | ✓ | ✓ |
| 75 kD | ✓ | ✓ | ✓ |
| 120 kD | X | X | X |
| 150 kD | X | X | X |

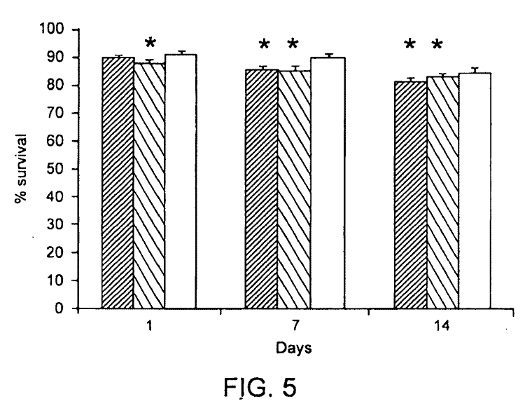

The difference in viability of human islets encapsulated in Ba X-Caps and Bi X-Caps as compared to APA microcapsules was shown to be a minor but statistically significant (p>0.05) amount (FIG. 5). This reduction in viability appears to occur during the encapsulation procedure as change in viability of human islets in Ba X-Caps and Bi X-Caps was not found to have a statistically significant difference from viability of human islets in APA microcapsules after fourteen days in culture (Table 3).

TABLE 3
Percent change in viability of human islets encapsulated in
barium x-caps, bismuth x-caps and APA controls from
1-7 days, 7-14 days and 1-14 days in culture.

| | Barium | Bismuth | Control |
|---|---|---|---|
| Day 1-7 | 4.0 ± 0.3* | 2.4 ± 1.2 | 1.8 ± 1.0 |
| Day 7-14 | 5.0 ± 1.0 | 2.9 ± 2.2 | 5.5 ± 1.7 |
| Day 1-14 | 8.8 ± 0.3 | 5.2 ± 1.4 | 7.5 ± 1.4 |

*statistically significant difference as compared to control.

The insulin secretory response of islets in Ba X-Caps and Bi X-Caps was compared against standard APA microcapsules. In order to finely detect any difference in insulin secretion from human islets in the various capsule types, encapsulated islets were incubated in solutions of 6, 7 and 8 mM glucose concentration. One day after encapsulation, no statistically significant difference (p>0.05) in insulin secretion was found in islets encapsulated in the various capsule types after incubation in 6 mM, 7 mM and 8 mM glucose solutions. Glucose responsiveness stimulation index as defined by increase in insulin secretion after changing from 6 mM to 8 mM glucose solution was found to be 1.76 for magnetocapsules, 1.69 for barium x-caps, 1.59 for bismuth capsules and 1.9 for APA microcapsules. To assess changes in insulin production over time, C-peptide secretion from human islets encapsulated in each capsule preparation was assessed over ninety minutes in an 8 mm glucose solution after 7 and 14 days in culture. The C-peptide secretion (ng/islet) from encapsulated islets at 7 and 14 days was found to be, respectively, 3.21 and 2.87 for Ba X-Caps, 3.23 and 2.95 for Bi X-Caps and 3.53 and 3.03 for APA microcapsules.

In additional to traditional classical hypothesis testing a more rigorous yet more informative statistical analysis was employed. Unlike classical hypothesis testing with a null hypothesis that two samples are the same, Bioequivajence (BE) testing uses the null hypothesis that two samples are different. The alternative hypothesis under BE testing, is that two samples differ by no more than some value theta. Theta is a value determined by the scientific community to be the maximum allowable difference between two samples, and still consider the samples to be bioequivalent. Because no value of theta has been established by the community, the theta that would be needed for each sample to be declared bioequivalent if TOST was run at an alpha level of 0.05 is reported here. Theta is reported as a percent difference from control. For 8 mM glucose solution, theta for Ba X-Caps to control (APA microcapsules) was 18.1 and Bi X-Caps to control was 32.3. For 6 mM glucose solution, theta for Ba X-Caps to control was 43.3 and Bi X-Caps to control was 47.3. For the magnitude change of C-peptide secretion from 6 to 8 mM glucose solution, the theta for Ba X-Caps to control was 32.9, and Bi X-Caps to control was 41.5.

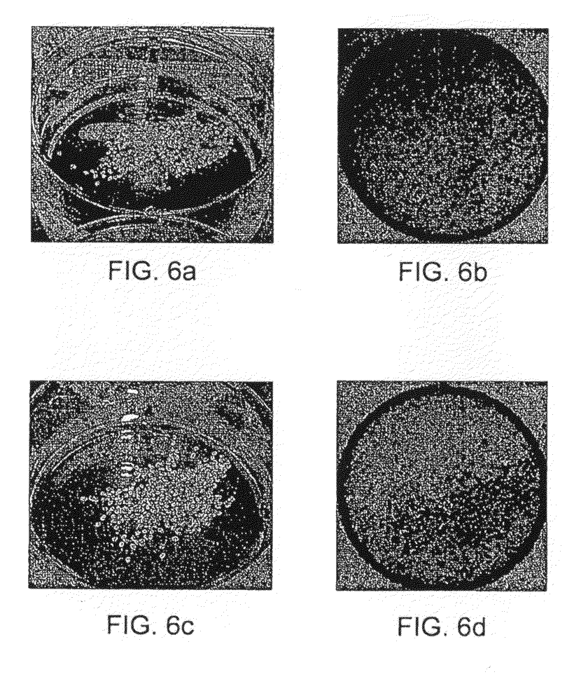

The visibility of contrast impregnated microcapsules permitted tracking of the implanted microcapsules without invasive surgery. Individual Ba X-Caps and Bi X-Caps could be visualized in vitro in normal saline in a multiwell plate (FIG. 6). Individual Ba X-Caps and Bi X-Caps could also be detected in vivo by standard fluoroscopy after transplantation into the peritoneal cavity of a mouse and after intramuscular injection into the hind limb of a rabbit. For both capsule types, real time imaging of delivery of capsules was possible under fluoroscopic guidance.

### Example 8

Fresh human cadaveric islets were provided by the National Islet Cell Resource Program and were encapsulated according to the procedure described herein. Microencapsulated islets were cultured in RPMI 1640 medium (Gibco), supplemented with 10% fetal calf serum and 1% penicillin/streptomycin/L-glutamine (all reagents from Sigma Co) in a humidified CO₂ incubator at 37° C. and a 5% CO₂ atmosphere. Microencapsulated islets were cultured in tissue culture plates and culture medium was replaced every three days.

Perfluorocarbon agents (PFCs) used were composed of perfluoro-15-crown-5 ether (PFPE, Exfluor Research) or perfluorooctylbromide (PFOB, Sigma Co.). The hydrophobic liquid PFC (1.97 g/mL for perfluorooctylbromide, 1.88 g/mL for perfluoropolyether) was then filtered through a 0.2 μm nylon filter (Acrodisc, Pall Corporation). The respective sterile-filtered PFC was then emulsified (20% vol/vol) in a mixture of 5% lecithin, 2% safflower oil and water by sonication at 40% power.

Fluorocapsules were formed using a solution of human cadaveric islets suspended in 2% w/v ultrapurifed Protanal HD Alginate (FMC Biopolymers, Norway) with 20% v/v emulsified PFOB or PFPE in conjugation with an electrostatic droplet generator. Alginate beads were transformed into alginate capsules by gelling in a 100 mM solution of CaCl₂. Microcapsules were washed with 0.9% saline and were subsequently suspended in a solution of 0.1% PLL allowing positively charged PLL to bind to the negatively charged alginate. Following PLL binding, microcapsules were suspended in 0.15% Keltone HVCR alginate (Monsanto, St. Louis, Mo.). Finally to remove any unbound alginate, microcapsules were washed with 0.9% saline.

To determine the permeability of PFC containing microcapsules compared to non-contrast containing capsules, microcapsule preparations were incubated with one of four fluorescently labeled lectins of varying molecular weight. Lectin incubation consisted of incubation with 15 L (1 mg/L) of FITC-Triticum vulgare (WGA, molecular mass=36 kDa), FITC-Maackia amurensis I (MAL-1, molecular mass=75 kDa), FITC-Ricinus communis (RCA-1, molecular mass=120 kDa), or FITC-Sambuca nigra (SNA, molecular mass=150 kDa). All lectins were obtained from EY Lab Inc. except FITC-Maackia amurensis I (Vector Laboratories). Capsules were incubated for 48 h at 4° C. on a mechanical rocker, after which they were examined microscopically (Olympus X51 and IX71 epifluorescence microscopes equipped with an Olympus DP-70 digital acquisition system) following embedding with Vectashield mounting medium (Vector, Burlingame, Calif.).

Following encapsulation, the viability of human islets was determined by a microfluorometric assay. Encapsulated islet cells were incubated with 10 mM Newport Green (NG, Sigma, St. Louis, Mo.) for 30 min and 5 mM propidium iodide (PI, Sigma, St. Louis, Mo.) for 10 min. NG was excited using the 500 nm laser line, and the emitted fluorescence was detected through a 535 nm long-pass filter. PI was excited using the 514 nm laser line, and the emitted fluorescence was detected through a 550 nm long-pass filter. Red fluorescent (PI) cells were counted as dead, and green fluorescent (NG) were counted as viable. In cases of dual partial red and green staining, cells were counted as dead. Seven representative microcapsules from three independent preparations each (total of 21) were randomly selected for cell viability determinations. Random selection of microcapsules was accomplished by vortexing each tube just prior to pipetting a 100 μL sample from the tube onto a glass slide.

A static incubation assay was used to assess the insulin secretion response of encapsulated human islets. One hundred encapsulated islets were placed in a culture insert (membrane pore diameter 12 m; Millicell PCF, Millipore, France) in 6 well plates. The insulin secretion was measured after 1.5 h in a solution of a specific glucose level. Specifically, a stepwise increase in glucose concentration from 3 mM to 8 mM D-glucose in RPMI 1640 medium was employed to assess the glucose responsiveness of encapsulated cells. Aliquots of the medium were stored at −80 C. The C-peptide content of the samples was determined with an enzyme linked immunosorbent assay (ultrasensitive human C-peptide ELISA, Alpco Diagnostics, Windham, N.H.); results (in ng/mL) were expressed as the means of three independent experiments. Insulin secretion assays were repeated at 7 days and 14 days following islet encapsulation.

For phantom creation, fluorocapsules were suspended in 4% gelatin. Specifically, a plastic mold was partially filled with warm gelatin solution and cooled until a solid state was obtained. Small indentations were made in the gelatin bed and a warm layer of gelatin was then poured over the solid layer. The appropriate number of fluorocapsules was then injected into the indentations in the hardened gelatin bed to create approximate point sources. The entire phantom was then cooled to achieve gelation.

For the PO₂ calibration, three 15 mL tubes (Falcon Co.) were filled with 1 mL of PFOB alginate that had been pre-gelled with exposure to a calcium chloride solution as described above. An MR-compatible fiberoptic oxygen sensor (Oxford Optronix, Oxford, England) was advanced into the alginate. The tube was then infused with different concentrations of nitrogen (PO₂=0 mm Hg), air (PO₂=160 mm Hg), or 100% oxygen (PO₂=760 mm Hg) to create varying oxygen concentrations as recorded by the fiberoptic sensor. The T1 value was then determined 5 times per tube at 9.4 T using the same MR sequence as for the in vivo studies.

MR imaging was performed using a 9.4 T MRI Scanner (Bruker BioSpin MRI GmbH), using a home-built RF solenoidal probe tunable to ¹⁹F and ¹H frequencies. A standard T₂ weighted spin echo (SE) pulse sequence was employed. Sixteen images were acquired for both anatomical proton and fluorine imaging using the SE parameters: TR/FE=1500/15 ms; FOV 3×3 cm; matrix 128×64 pixels; slice thickness 1 mm; NA=1, total scan time 96 seconds. Segmentation and 3D reconstruction were done using the imaging software Amira (Mercury Computer Systems).

Images were obtained using a Gamma Medica XSPECT scanner. CT subjects were placed on an animal bed and anesthetized with 2.5% isoflurane flowing at 0.5 L/min throughout the imaging with exposure to radiation limited to a maximum of 30 minutes. For each scan, 1024 projections with 1024×1024 pixels were obtained at different angles of view between 0° and 360°. Acquisition time for each view was 1 second. Scanning was performed in a clockwise direction with an X-ray tube to detector distance of 269 mm and an X-ray tube to COR distance of 225 mm. Images were obtained in rotation steps of 0.703° with respective voltage and current of 50 kVp and 600 mA. Segmentation and 3D reconstruction were done using the imaging software Amira.

Fifteen mice were transplanted with encapsulated human islets cells into the peritoneal cavity and the other 15 mice were transplanted with empty microcapsules (no islets). Before the transplantation, encapsulated islets were cultured overnight as described above. Under general isoflurane anesthesia, mice received a single IP transplant of 6,000 empty microcapsules or microcapsules containing ˜1 human islet.

Every 4-5 days blood samples were taken via the tail vein for measurement of serum human C-peptide over a 6-week period using an “ultrasensitive” human C-peptide ELISA kit (Alpco Diagnostics, Windham, N.H.). Samples were stored at −80° C. for insulin quantification. To assess the MR detectability of fluorocapsules in mice, 500 fluorocapsules were transplanted IP. Immediately after injection, MR imaging was performed at 9.4 T.

Experiments were performed on eight healthy swine (40-45 kg) that were sedated with 1 ml/50 lbs of telazol/ketamine/xylazine (100/10/100 mg/ml). Induction was followed by endotracheal intubation and mechanical ventilation with oxygen and 1-2% isoflurane anesthesia. After endotracheal intubation, mechanical ventilation was started. To reduce the effect of mechanical ventilation on the image quality, swine were ventilated with small tidal volumes (350-400 mL) at a rate of 26 breaths per minute. A 7-F arterial sheaths (Cordis, Miami, Fla.) was placed in the common femoral arteries through ultrasound guidance and used for advancement of intraarterial catheters. A 5-F pigtail catheter (Cook, Bloomington, Ind.) was inserted into the aorta at the level of the diaphragm under fluoroscopic guidance. DSA was then performed by injecting 10 mL of iodinated radiographic contrast material (diatrizoate meglumine, Hypaque; Nycomed, Princeton, N.J.) to define location and number of the renal arteries. The renal arteries were subsequently catheterized with a 5-F Cobra catheter (Cook), and bilateral selective renal DSA was performed to verify vessel patency and assess baseline status of both kidneys.

All CT examinations were performed on a multidetector CT scanner with 120 kV tube voltage and 165 mAs (Somatom Volume Zoom, Siemens). The scanning range covered the diaphragm to the pelvic floor.

Sonography was performed with a 6.5EC10 probe on a Sonoline Elegra system (Siemens Medical Systems, Issaquah, Wash.). Grayscale imaging was performed with a center probe frequency of 5.14-6.00 MHz, a dynamic range of 55 dB, and a persistence setting of two. Gray-scale gain was adjusted for baseline imaging. PFOB fluorocapsules were then transplanted via direct injection with a 20 gauge needle into the kidney of a swine and images were digitally captured without altering baseline gray-scale gain.

Statistical analysis was conducted using a Students T-test with a significance level P<0.05. Data were also analyzed using the bioequivalence (BE) test. The test was performed using the Two-One Sided T-test approach (TOST). In a BE test, the null hypothesis is that two groups differ by an amount θ or more. In TOST, the null hypothesis is rejected and two groups are declared bioequivalent at the type I error rate θ if a (1-2θ) confidence interval is contained in (−θ, θ). Because no θ value has been established for declaring bioequivalence in islet cell viability, the lowest value that would allow the two samples to be declared bioequivalent is reported, with θ being reported as a percent difference from control. All statistical analysis was done using the statistical software R.

The relaxation rate signal (1/T₁) of the PFOB capsules consistently demonstrated a roughly linear pattern with oxygen concentration.

Differences in viability of human islets encapsulated in PFOB, PFPE and non-PFC containing microcapsules were assessed at days 1, 7, and 14. The percentage viability of PFC encapsulated islets was increased as compared to non-PFC encapsulated islets on days 7 and 14 (p>0.05). The BE value, 0, for all comparisons was less than 5%. The glucose stimulation index, defined as the ratio of insulin secretion at 8 mM glucose to insulin secretion at 3 mM glucose, was measured after 1, 7, and 14 days. For each day, there was no statistically significant difference between the PFC containing and non-PFC containing microcapsules (p>0.05).

The PFCs used are hydrophobic and insoluble, and incorporation into a hydrophilic alginate hydrogel requires incorporation of PFCs into micelles. Examination of permeability to fluorescent lectins of varying molecular weights revealed no appreciable difference in perm selectivity. Permeability of standard APA capsules and fluorocapsules was determined by incubation for 48 hours with fluorescently labeled lectins of varying molecular weight. APA capsules and fluorocapsules were both found to be permeable to WGA, MW: 36 kD and MAL-I, MW: 75 kD but impermeable to RCA-I, MW: 120 kD and SNA, MW: 150 kD. As capsules are permeable to fluorescent lectins <75 kD but were found to be impermeable to lectins >120 kD, they are capable of blocking antibody penetration (immuno-isolation) while allowing inflow of nutrients and secretion of therapeutic factors by encapsulated cells.

After transplantation of 6,000 PFOB or PFPE encapsulated human islets into the peritoneal cavity of mice, serum human C-peptides levels ranged from 15.52 and 23.59 μmol/L for PFPE transplanted mice (n=15) and 24.19 to 37.66 μmol/L for PFOB transplanted mice (n=15) microcapsules over the fifty days in which mice were observed. Control mice transplanted with empty PFPE (n=2) or PFOB (n=2) microcapsules confirmed species specificity of ELISA as human C-peptide levels were non-detectable.

In vitro ¹⁹F MR imaging of 350 μm fluorocapsules demonstrated the ability to detect single capsules at 11.7 T. Using high-resolution ¹⁹F MRI (Bruker 4.7 T animal scanner), following transplantation into the peritoneal cavity of mice, fluorocapsules were identifiable and when overlaid on anatomical ¹H MRI scans, capsules were easily distinguishable from soft tissue. With imaging on an animal grade CT, individual capsules were visible in vitro and in vivo when transplanted into the peritoneal cavity of a mouse. On a clinical grade CT scanner, groups of PFOB caps were clearly distinguished in vitro and after transplantation into the kidney of a swine. Additionally, individual fluorocapsules in both phantoms and after transplantation into the kidney of a swine were visible under ultrasound).

### Example 9

Recent studies suggest that angiogenesis following stem cell administration offers a possible therapy to improve the clinical outcome of peripheral arterial disease (PAD). However, this approach has been hampered by the inability to determine whether cells reach their target and rapid cellular destruction. An allogeneic mesenchymal stem cell (MSCs) encapsulation method was developed that enables x-ray visualization and protection of allogeneic cells from immune destruction. The first use of this x-ray visible stem cell encapsulation technique is described in a rabbit model of PAD.

METHODS: The classical method of alginate encapsulation was modified by the addition of barium sulfate (10% w/w) to Protanal HF alginate (2.0%) and Poly-L-lysine (0.05%) to fabricate microcapsules (XCaps) containing MSCs from male New Zealand White (NZW) rabbits. MSC viability after encapsulation was evaluated in vitro. Twenty-four hours after creation of hindlimb ischemia using a percutaneous coil technique, female NZW rabbits (n=13) were randomized to receive, intramuscularly, either: five thousand XCaps with MSCs (n=5), without MSCs (n=5), naked MSCs (n=1), or sham (n=2) injections. XCaps visibility was assessed via x-ray fluoroscopy immediately and at 2 weeks post-injection. Angiogenesis was determined using digital subtraction angiography at 2 weeks and after euthanasia using immunohistochemistry.

RESULTS: MSCs viability was 78±4.3% at day 1 and remained at 57±4.58% after 1 week following encapsulation. XCaps both with and without MSCs were visible immediately and at 2 weeks post-injection. Collateral formation was robust on X-ray angiography at 2 weeks and was consistent with histological findings.

CONCLUSIONS: XCaps technology offers a new approach for immediate visualization of stem cell injection success using conventional X-ray fluoroscopy and protection from immune destruction. In addition, microencapsulation provides a means to enhance cellular retention and overcome early destruction due to immune rejection. Thus, this novel method to enable visualization and enhance engraftment of stem cells shows considerable potential in future clinical applications.

### Example 10

Cell Culture. Fresh human cadaveric islets were provided by the Joslin Diabetes Research Center (National Islet Cell Resource Program). Average purity and viability were 90% and 85%. For microencapsulated cells, groups of 100 microcapsules each containing <1 islet were cultured in multi-well plates. Murine βTC-6 insulinoma cells (ATCC) were grown in medium containing 5.5 mM glucose.

Magnetoencapsulation. MC synthesis is based on a one-step modification (i.e., Feridex® addition) of the Lim-Sun method. This modification uses an electrostatic (van de Graaff) droplet generator, producing smaller, stronger, and more uniform capsules compared to the older air-jet technique. Before encapsulation, human cadaveric islets were passed through a 20 g needle. Cells, adjusted to 400 islet equivalents/ml or 1.5×10⁷ cells/ml (OTC-6) were suspended in 2% w/v ultrapurified sodium Protanal®-HF alginate (FMC Biopolymers) and 20% vol/vol Feridex® (Berlex Laboratories). This solution was passed through a needle at 200 μl/min using a nanoinjector pump. Droplets, representing islet cells surrounded by the first layer of alginate, were collected in a Petri dish containing 100 mM CaCl₂ in 10 mM HEPES, and washed three times. Gelled droplets were suspended in 0.05% poly-L-lysine (Sigma, Mw=22-24 kDa) for 5 min to crosslink alginate and Feridex®. Droplets were washed and resuspended in 0.15% Keltone HVCR alginate (Monsanto) for 5 min, and washed again. For capsule rupture, MCs were manually agitated in a 50 ml conical tube filled with 1 mm glass beads.

In vitro characterization of MCs. The presence of Feridex® in MCs was assessed with Prussian Blue staining and a spectrophotometric, Ferrozin-based iron assay of acid-digested samples. Immunostaining using a dextran-specific antibody (Stemcell Technologies) was used to visualize dextran-coated Feridex® particles within MCs.

Following magnetoencapsulation, cell viability was determined using a microfluorometric assay. Encaspulated cells were incubated with 10 mM Newport Green (NG, Sigma) for 30 min and 5 mM Propidium Iodide (PI, Sigma) for 10 min. Seven representative microcapsules from three independent preparations each (21 total) were randomly selected. For capsule permeability measurements, MCs were incubated with one of four fluorescently labeled lectins of varying molecular weight as described herein.

A static incubation assay was used to assess the insulin secretion response (Supplementary Information). For assessment of MR contrast, MCs were suspended in 2% agarose at a density of 50 capsules/ml gel, with phantom imaging performed at 3 T.

Mice studies. For the induction of diabetes, C57/BL mice (Charles River, n=30) were given streptozotocin IV at 185 mg/kg. Mice were considered diabetic if they had three consecutive, non-fasting blood glucose levels >20 mM, as measured using a glucometer (Lifescan/Johnson and Johnson). Fifteen mice were transplanted with MC βTC-6 cells into the peritoneal cavity and the other 15 mice received empty MCs (no cells). Under isoflurane anesthesia, mice received a single IP transplant of 6,000 empty MCs or 6,000 MCs containing 500 cells each (total of 3×10⁶ cells). Every 2-3d, body weight was measured and blood samples taken for blood glucose measurements. To assess MR detectability of MCs in mice, 500 MCs were transplanted IP. Immediately after injection, MRI was performed at 9.4 T.

Swine studies. Ten healthy swine (40-45 kg) were used. Using ultrasound guidance, percutaneous access into the right femoral vein was achieved with an 11F sheath Animals were transferred to the MR suite, and a sheath with a (MR-visible) nitinol marker was advanced into the IVC. An intravascular puncture of the portal vein was performed using a custom-built, MR-trackable needle. An access puncture from the IVC to the portal vein was made below the splenic vein using real-time MR guidance. A 0.038 nitinol guidewire (Nitrex) was advanced into the portal vein, and the puncture needle was exchanged for an 8F catheter with a nitinol marker on the distal tip to allow for MR visualization. The 8F catheter was advanced under MR fluoroscopy into the portal vein for infusion of 40,000 MCs. MRI was performed immediately and at 3 wk following MC transplantation. In two swine, a larger dose of 140,000 MCs in a packed volume of 6 mls saline was given, and liver function (blood) tests and portal pressure measurements (pressure transducer) were obtained over 4 wks. In one swine, 40,000 human MC islets were injected and blood drawn before and at 1, 2, and 3 wks after transplantation. Specific human C-peptide levels were measured using an ELISA (Alpco Diagnostics).

Ex vivo imaging and histological correlation. After euthanasia, the liver was harvested, fixed with 4% paraformaldehyde, and suspended in a styrofoam box filled with 3% w/v gelatin. MRI was performed at 3 T. The liver was sliced into 1 cm transverse sections and processed stained with Prussian Blue.

Statistical analysis. A student's T-test with a significance level of P<0.05 was used. Data were also analyzed using the bioequivalence (BE) test, with a Two-One-Sided T-test approach (TOST). All analyses were performed using the software R.

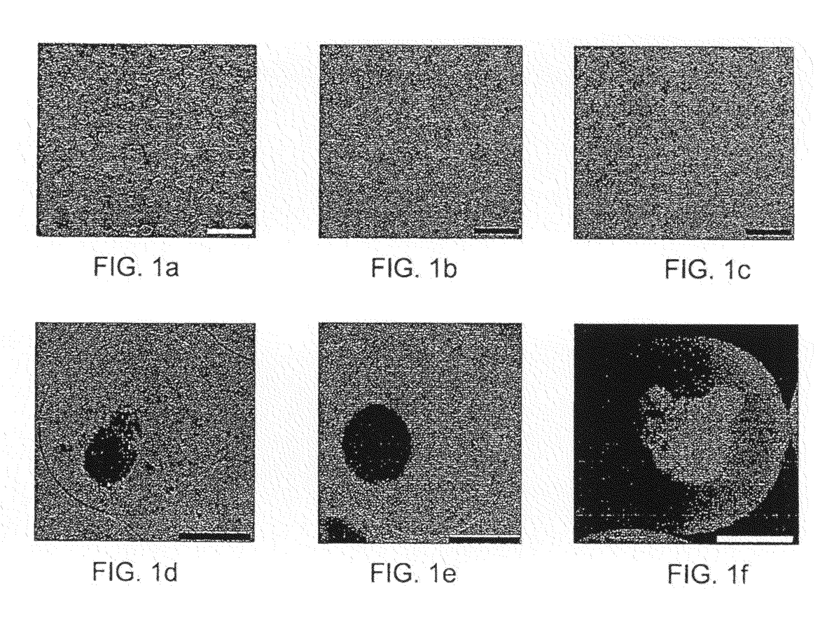

Magnetoencapsulation. The macroscopic appearance of MCs (FIG. 1a, c) and microscopic appearance of encapsulated cells (FIG. 1d-b) showed uniformity in size (˜350 μm in diameter). MC preparations were stable for at least 18 months. When prepared with alginate containing 20% v/v Feridex® the iron concentration as determined by Ferrozin assay is 80.8±4.9 ng Fe per capsule. MCs containing human islets (FIG. 1e) exhibited a characteristic Feridex®-like color. Dextran-specific immunostaining (FIG. 1f, g) demonstrated that Feridex® labeling was uniform without particle clustering.

In vitro comparisons did not reveal differences in capsule permeability, islet cell viability, or insulin secretory response. Both unlabeled microcapsules and MCs were permeable to lectins ≦75kDa, but impermeable to lectins ≧120 kDa, ensuring blockage of antibodies while allowing diffusion of insulin (˜5 kDa) and nutrients. Magnetoencapsulated βTC6 cell viability (FIG. 1e) was 94% and 82% at 3 and 6 weeks, respectively, similar to unlabeled capsules (96% and 81% at 3 and 6 weeks).

MC human islets retain functional properties in vitro. The viability of MC human islets also did not differ from encapsulation without Feridex®. One day post-encapsulation, no difference in insulin secretion existed between MCs and non-magnetic capsules (p<0.05). The glucose responsiveness stimulation index was 3.36±0.21 and 3.50±0.38 for MC and unlabeled capsules, respectively. The insulin secretory response of MC human islets was assessed over 15 days. Using the FDA-approved bioequivalence test (TOST, threshold=5%, α=0.05), insulin secretion from MC islets was compared to islets encapsulated without Feridex®. Except for a small decrease at day 3, MC islet insulin secretion was bioequivalent to secretion by islets in unlabeled capsules, ranging between 2-2.5 ng insulin per islet. Thus, Feridex® incorporation does not alter capsule “porosity” and insulin diffusion.

MRI properties of MCs. With 81 ng Fe per capsule, a clear MRI depiction of single capsules in agarose phantoms and mice could be obtained. Using 3D inversion-recovery on-resonance (IRON) positive contrast MRI, the capsule surface of single-MCs could be selectively enhanced. Using conventional MRI sequences, MR properties changed substantially following capsule rupture, with a 72% loss of the hypointense signal.

MC insulinoma cells restore normal glycemia in diabetic mice. MCs (n=6000, each containing 500 βTC6 cells for 3×10⁶ cells total) were transplanted IP in streptozotocin (STZ)-induced diabetic mice (n=15). Blood glucose levels normalized to about <100 mg/dl by 1 week of transplantation and remained constant throughout 8 weeks. In contrast, 9 out of 15 non-transplanted animals died while the surviving mice remained hyperglycemic. PTC6 MC-transplanted but not untransplanted animals showed increasing net weight gain. Murine insulin levels were significantly increased at 4 and 8 weeks post-PTC6 MC engraftment, but not in controls.

MR-guided delivery, tracking, and functionality of MCs in swine. Using an MR-compatible catheter, 40,000 MCs were infused into the portal vein of swine. This allowed real-time monitoring of correct catheter positioning and initial liver engraftment on a 1.5 T clinical scanner. The needle was actively tracked as it traversed the inferior vena cava (IVC) toward the portal vein. Following precise infusion, MCs were clearly visualized as hypointensities, representing capsule distribution within the entire liver. MC distribution was predominantly in the liver periphery with central sparing, correlating to normal portal vein flow patterns. Follow-up MRI at 3 weeks demonstrated no changes in MR-appearance or health complications. Even after a larger dose of 140,000 MCs, blood bilirubin, alkaline phosphatase, aspartate aminotransferase, alanine aminotransferase and platelet counts were within normal values over 4 weeks post-transplantation. A mild transient increase in portal pressure occurred immediately after injection, with a return to near-baseline values at 30 min post-injection, followed by normal pre-injection values persisting for at least 4 weeks. Grafted human MC islets secreted insulin, with circulating C-peptide values of 0.27-0.38 ng/ml over 3 weeks.

Following in vivo MRI, the liver was imaged ex vivo using a 3 T clinical scanner. T2*-weighted MRI revealed strong hypointensities and confirmed the in vivo findings, with MCs lodged in the distal microvasculature of the liver. A 3D reconstruction of the in vivo MRI showed distribution in the distal vasculature in all three dimensions throughout the entire liver.

The foregoing is illustrative of the present invention, and is not to be construed as limiting thereof. The invention is defined by the following claims, with equivalents of the claims to be included therein

1. A microcapsule for implantation into a mammalian body, comprising:
a) at least one cell and/or bioactive agent; and
b) a biocompatible semi-permeable membrane encapsulating the at least one cell and/or bioactive agent,
wherein the biocompatible semi-permeable membrane comprises:
at least one polycationic polymer region,
at least one alginate polymer region, and
a paramagnetic or superparamagnetic metal that does not participate in the crosslinking of the alginate polymer.

2. The microcapsule of claim 1, wherein the polycationic polymer is poly-L-lysine.

3. The microcapsule of claim 1, wherein the paramagnetic or superparamagnetic metal is selected from the group consisting of iron, gadolinium, manganese, dysprosium and any combination thereof.

4. The microcapsule of claim 1, wherein the paramagnetic or superparamagnetic metal is iron, and wherein the iron is present in the microcapsule as a ferum-oxide.

5. The microcapsule of claim 4, wherein the ferum-oxide is derived from a Feridex® or Resovist® aqueous colloidal solution.

6. The microcapsule of claim 1, wherein the at least one cell comprises an islet cell.

7. A microcapsule for implantation into a mammalian body, comprising:
(i)
a) at least one cell and/or bioactive agent; and
b) a biocompatible semi-permeable membrane encapsulating the at least one cell and/or bioactive agent,
wherein the biocompatible semi-permeable cell membrane comprises:
at least one polycationic polymer region,
at least one alginate polymer region, and a radiopaque contrast agent;
or
(ii)
a) at least one cell and/or bioactive agent; and
b) a biocompatible semi-permeable membrane encapsulating the at least one cell and/or bioactive agent, wherein the biocompatible semi-permeable cell membrane comprises:
at least one polycationic polymer region; and
at least one alginate Polymer region, and
a fluorocarbon.

8-17. (canceled)

18. A microcapsule for implantation into a mammalian body, comprising:
a) at least one cell and/or bioactive agent; and
b) a biocompatible semi-permeable alginate layer encapsulating the at least one cell and/or bioactive agent, wherein the biocompatible semi-permeable alginate layer comprises a paramagnetic or superparamagnetic metal that does not participate in the crosslinking of the alginate layer.

19. The microcapsule of claim 18, wherein the paramagnetic or superparamagnetic metal is selected from the group consisting of iron, gadolinium, manganese, dysprosium and any combination thereof.

20-22. (canceled)

23. A composition comprising the microcapsule according to claim 1 in a pharmaceutically acceptable carrier.

24. A composition comprising the microcapsule according to claim 7 in a pharmaceutically acceptable carrier.

25. (canceled)

26. A composition comprising the microcapsule according to claim 18 in a pharmaceutically acceptable carrier.

27. A method of delivering at least one cell and/or bioactive agent to a mammal, comprising introducing the microcapsule according to claim 1 into the mammal.

28-31. (canceled)

32. A method of synthesizing an MRI-detectable microcapsule, comprising:
(i)
a) forming a droplet comprising:
a cell and/or bioactive agent,
an alginate polymer that is not crosslinked with a paramagnetic or superparamagnetic metal, and
at least one of a paramagnetic or a superparamagnetic metal;
b) adding a crosslinking agent to crosslink the alginate polymer;
c) introducing the crosslinked droplet to a polycationic polymer solution; and
d) introducing the polycationic polymer-treated crosslinked droplet to an alginate polymer solution;
or
(ii)
a) forming a droplet comprising:
a cell and/or bioactive agent, and
an alginate polymer that is not crosslinked with a paramagnetic or superparamagnetic metal;
b) adding a crosslinking agent to crosslink the alginate polymer;
c) introducing the crosslinked droplet to a polycationic polymer solution: and
d) introducing the polycationic polymer-treated crosslinked droplet to an alginate polymer solution.

33-42. (canceled)

43. A method of synthesizing a radiopaque microcapsule comprising:
(i)
a) forming a droplet comprising a cell and/or bioactive agent, an alginate polymer and a radiopaque contrast agent;
b) adding a crosslinking agent to crosslink the alginate polymer;
c) introducing the crosslinked droplet to a polycationic polymer solution; and
d) introducing the polycationic polymer-treated crosslinked droplet to an alginate polymer solution;
or
(ii)
a) forming a droplet comprising:
a cell and/or bioactive agent,
an alginate Polymer that is not crosslinked with a paramagnetic or superparamagnetic metal, and
at least one of a paramagnetic or a superparamagnetic metal: and
b) adding a crosslinking agent to crosslink the alginate polymer.

44-52. (canceled)

53. A method of embolizing a vascular site through physical obstruction, comprising introducing into the vascular site one or more microcapsules comprising a biocompatible semi-permeable membrane, wherein the biocompatible semi-permeable membrane comprises:
at least one polycationic polymer region,
at least one alginate polymer region, and
a paramagnetic or superparamagnetic metal that does not participate in the crosslinking of the alginate polymer.

54. A kit comprising:
a) the composition according to claim 23;
b) a syringe; and optionally
c) instructions for using the syringe to inject the composition into a mammal.

55. An MRI system for MRI imaging of the microcapsule according to claim 1, comprising:
a) an MRI scanner;
b) a receiver configured to detect a magnetic resonance signal produced from the microcapsule; and
c) a display in communication with the MRI scanner configured to display in vivo images of the microcapsules in target tissue.

56. (canceled)

57. An apparatus for imaging the microcapsule according to claim 1 comprising:
a) an X-ray source, and
b) an X-ray detection device and output circuit that generates visual data associated with the location of the microcapsule in a position in the body.
